SEQUENCE LISTING (40 sequences)

SEQ ID NO: 1 - DNA, 27 bp, Artificial Sequence
  Description of Artificial Sequence PCR Primer
ccatcctaatacgactcactatagggc

SEQ ID NO: 2 - DNA, 27 bp, Artificial Sequence
  Description of Artificial Sequence PCR Primer
ggatcccgggtggatggtgggaagatg

SEQ ID NO: 3 - DNA, 28 bp, Artificial Sequence
  Description of Artificial Sequence PCR Primer
ggatcccgggccagtggatagacagatg

SEQ ID NO: 4 - DNA, 26 bp, Artificial Sequence
  Description of Artificial Sequence PCR Primer
ggatcccgggagtggatagaccgatg

SEQ ID NO: 5 - DNA, 394 bp, Mus sp.
  CDS (1)..(393)
atgaagttgcctgttaggctgttggtgctgatgttctggattcctgcgtccagcagtgatgttgtgatgacccaaactccactctccctgcctgtcagtcttggagatcaagcctccatctcttgcagatctagtcagagccttctacacagtaaaggaaacacctatttacaatggtacctacagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgtccggaggggggaccaagctggaaataaaac

SEQ ID NO: 6 - protein, 131 aa, Mus sp.
MKLPVRLLVLMFWIPASSSDVVMTQTPLSLPVSLGDQASISCRSSQSLLHSKGNTYLQWYLQKPGQSPKLLIYKVSNRFSGVPDRFSGSGSGTDFTLKISRVEAEDLGVYFCSQSTHVPYTSGGGTKLEIK

SEQ ID NO: 7 - DNA, 409 bp, Mus sp.
  CDS (1)..(408)
atggaatggagctggatatttctcttcctcctgtcaggaactgcaggtgtccactcccaggtccagctgcagcagtctggacctgacctggtaaagcctggggcttcagtgaagatgtcctgcaaggcttctggatacaccttcgttaaccatgttatgcactgggtgaagcagaagccagggcagggccttgagtggattggatatatttatccttacaatgatggtactaagtacaatgagaagttcaagggcaaggccacactgacttcagagaaatcctccagcgcagcctacatggagctcagcagcctggcctctgaggactctgcggtctactactgtgcaagagggggttactatagttacgacgactggggccaaggcaccactctcacagtctcctcag

SEQ ID NO: 8 - protein, 136 aa, Mus sp.
MEWSWIFLFLLSGTAGVHSQVQLQQSGPDLVKPGASVKMSCKASGYTFVNHVMHWVKQKPGQGLEWIGYIYPYNDGTKYNEKFKGKATLTSEKSSSAAYMELSSLASEDSAVYYCARGGYYSYDDWGQGTTLTVSS

SEQ ID NO: 9 - DNA, 394 bp, Mus sp.
  CDS (1)..(393)
atgaagttgcctgttaggctgttggtgctgatgttctggattcctggttccagcagtgatgttgtgatgacccaaagtccactctccctgcctgtcagtcttggagatcaagcctccatctcttgcagatcaagtcagagccttgtgcacagtaatggaaagacctatttacattggtacctgcagaagccaggccagtctccaaaactcctgatctacaaagtttccaaccgattttctggggtcccagacaggttcagtggcagtggatcagtgacagatttcacactcatgatcagcagagtggaggctgaggatctgggagtttatttctgctctcaaagtacacatgttccgtacacgttcggaggggggaccaagctggaaataaaac

SEQ ID NO: 10 - protein, 131 aa, Mus sp.
MKLPVRLLVLMFWIPGSSSDVVMTQSPLSLPVSLGDQASISCRSSQSLVHSNGKTYLHWYLQKPGQSPKLLIYKVSNRFSGVPDRFSGSGSVTDFTLMISRVEAEDLGVYFCSQSTHVPYTFGGGTKLEIK

SEQ ID NO: 11 - DNA, 409 bp, Mus sp.
  CDS (1)..(408)
atggaatggagctggatatttctcttcctcctgtcaggaactgcaggtgtccactcccaggtccagctgcagcagtctggacctgaactggtaaagcctggggcttcagtgaagatgtcctgcaaggcttctggatacaccttcgctaaccatgttattcactgggtgaagcagaagccagggcagggccttgagtggattggatatatttatccttacaatgatggtactaagtataatgagaagttcaaggacaaggccactctgacttcagacaaatcctccaccacagcctacatggacctcagcagcctggcctctgaggactctgcggtctattactgtgcaagagggggttactatacttacgacgactggggccaaggcaccactctcacagtctcctcag

SEQ ID NO: 12 - protein, 136 aa, Mus sp.
MEWSWIFLFLLSGTAGVHSQVQLQQSGPELVKPGASVKMSCKASGYTFANHVIHWVKQKPGQGLEWIGYIYPYNDGTKYNEKFKDKATLTSDKSSTTAYMDLSSLASEDSAVYYCARGGYYTYDDWGQGTTLTVSS

SEQ ID NO: 13 - DNA, 32 bp, Artificial Sequence
  Description of Artificial Sequence PCR Primer
cccaagcttccaccatgaagttgcctgttagg

SEQ ID NO: 14 - DNA, 32 bp, Artificial Sequence
  Description of Artificial Sequence PCR Primer
cccaagcttccaccatggaatggagctggata

SEQ ID NO: 15 - DNA, 34 bp, Artificial Sequence
  Description of Artificial Sequence PCR Primer
cgcggatccactcacgttttatttccagcttggt

SEQ ID NO: 16 - DNA, 34 bp, Artificial Sequence
  Description of Artificial Sequence PCR Primer
cgcggatccactcacctgaggagactgtgagagt

SEQ ID NO: 17 - DNA, 30 bp, Artificial Sequence
  Description of Artificial Sequence PCR Primer
catgccatggcgcaggtccagctgcagcag

SEQ ID NO: 18 - DNA, 27 bp, Artificial Sequence
  Description of Artificial Sequence PCR Primer
accaccacctgaggagactgtgagagt

SEQ ID NO: 19 - DNA, 27 bp, Artificial Sequence
  Description of Artificial Sequence PCR Primer
gtctcctcaggtggtggtggttcgggt

SEQ ID NO: 20 - DNA, 27 bp, Artificial Sequence
  Description of Artificial Sequence PCR Primer
cacaacatccgatccgccaccacccga

SEQ ID NO: 21 - DNA, 27 bp, Artificial Sequence
  Description of Artificial Sequence PCR Primer
ggcggatcggatgttgtgatgacccaa

SEQ ID NO: 22 - DNA, 57 bp, Artificial Sequence
  Description of Artificial Sequence PCR Primer
ccggaattctcattatttatcgtcatcgtctttgtagtcttttatttccagcttggt

SEQ ID NO: 23 - DNA, 45 bp, Artificial Sequence
  Description of Artificial Sequence Plasmid pSC-DP1 comprising a DNA sequence coding a linker region
ggtggtggtggttcgggtggtggtggttcgggtggtggcggatcg

SEQ ID NO: 24 - protein, 15 aa, Artificial Sequence
  Description of Artificial Sequence Peptide linker
GGGGSGGGGSGGGGS

SEQ ID NO: 25 - DNA, 828 bp, Mus sp.
  CDS (1)..(822)
atgaaatacctattgcctacggcagccgctggattgttattactcgctgcccaaccagccatggcgcaggtccagctgcagcagtctggacctgacctggtaaagcctggggcttcagtgaagatgtcctgcaaggcttctggatacaccttcgttaaccatgttatgcactgggtgaagcagaagccagggcagggccttgagtggattggatatatttatccttacaatgatggtactaagtacaatgagaagttcaagggcaaggccacactgacttcagagaaatcctccagcgcagcctacatggagctcagcagcctggcctctgaggactctgcggtctactactgtgcaagagggggttactatagttacgacgactggggccaaggcaccactctcacagtctcctcaggtggtggtggttcgggtggtggtggttcgggtggtggcggatcggatgttgtgatgacccaaactccactctccctgcctgtcagtcttggagatcaagcctccatctcttgcagatctagtcagagccttctacacagtaaaggaaacacctatttacaatggtacctacagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgtccggaggggggaccaagctggaaataaaagactacaaagacgatgacgataaataatga

SEQ ID NO: 26 - protein, 274 aa, Mus sp.
MKYLLPTAAAGLLLLAAQPAMAQVQLQQSGPDLVKPGASVKMSCKASGYTFVNHVMHWVKQKPGQGLEWIGYIYPYNDGTKYNEKFKGKATLTSEKSSSAAYMELSSLASEDSAVYYCARGGYYSYDDWGQGTTLTVSSGGGGSGGGGSGGGGSDVVMTQTPLSLPVSLGDQASISCRSSQSLLHSKGNTYLQWYLQKPGQSPKLLIYKVSNRFSGVPDRFSGSGSGTDFTLKISRVEAEDLGVYFCSQSTHVPYTSGGGTKLEIKDYKDDDDK

SEQ ID NO: 27 - DNA, 31 bp, Artificial Sequence
  Description of Artificial Sequence PCR Primer
acgcgtcgactcccaggtccagctgcagcag

SEQ ID NO: 28 - DNA, 18 bp, Artificial Sequence
  Description of Artificial Sequence PCR Primer
gaaggtgtatccagaagc

SEQ ID NO: 29 - DNA, 819 bp, Mus sp.
  CDS (1)..(813)
atgggatggagctgtatcatcctcttcttggtagcaacagctacaggtgtcgactcccaggtccagctgcagcagtctggacctgacctggtaaagcctggggcttcagtgaagatgtcctgcaaggcttctggatacaccttcgttaaccatgttatgcactgggtgaagcagaagccagggcagggccttgagtggattggatatatttatccttacaatgatggtactaagtacaatgagaagttcaagggcaaggccacactgacttcagagaaatcctccagcgcagcctacatggagctcagcagcctggcctctgaggactctgcggtctactactgtgcaagagggggttactatagttacgacgactggggccaaggcaccactctcacagtctcctcaggtggtggtggttcgggtggtggtggttcgggtggtggcggatcggatgttgtgatgacccaaactccactctccctgcctgtcagtcttggagatcaagcctccatctcttgcagatctagtcagagccttctacacagtaaaggaaacacctatttacaatggtacctacagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgtccggaggggggaccaagctggaaataaaagactacaaagacgatgacgataaataatga

SEQ ID NO: 30 - protein, 271 aa, Mus sp.
MGWSCIILFLVATATGVDSQVQLQQSGPDLVKPGASVKMSCKASGYTFVNHVMHWVKQKPGQGLEWIGYIYPYNDGTKYNEKFKGKATLTSEKSSSAAYMELSSLASEDSAVYYCARGGYYSYDDWGQGTTLTVSSGGGGSGGGGSGGGGSDVVMTQTPLSLPVSLGDQASISCRSSQSLLHSKGNTYLQWYLQKPGQSPKLLIYKVSNRFSGVPDRFSGSGSGTDFTLKISRVEAEDLGVYFCSQSTHVPYTSGGGTKLEIKDYKDDDDK

SEQ ID NO: 31 - DNA, 828 bp, Mus sp.
  CDS (1)..(822)
atgaaatacctattgcctacggcagccgctggattgttattactcgctgcccaaccagccatggcgcaggtccagctgcagcagtctggacctgaactggtaaagcctggggcttcagtgaagatgtcctgcaaggcttctggatacaccttcgctaaccatgttattcactgggtgaagcagaagccagggcagggccttgagtggattggatatatttatccttacaatgatggtactaagtataatgagaagttcaaggacaaggccactctgacttcagacaaatcctccaccacagcctacatggacctcagcagcctggcctctgaggactctgcggtctattactgtgcaagagggggttactatacttacgacgactggggccaaggcaccactctcacagtctcctcaggtggtggtggttcgggtggtggtggttcgggtggtggcggatcggatgttgtgatgacccaaagtccactctccctgcctgtcagtcttggagatcaagcctccatctcttgcagatcaagtcagagccttgtgcacagtaatggaaagacctatttacattggtacctgcagaagccaggccagtctccaaaactcctgatctacaaagtttccaaccgattttctggggtcccagacaggttcagtggcagtggatcagtgacagatttcacactcatgatcagcagagtggaggctgaggatctgggagtttatttctgctctcaaagtacacatgttccgtacacgttcggaggggggaccaagctggaaataaaagactacaaagacgatgacgataaataatga

SEQ ID NO: 32 - protein, 274 aa, Mus sp.
MKYLLPTAAAGLLLLAAQPAMAQVQLQQSGPELVKPGASVKMSCKASGYTFANHVIHWVKQKPGQGLEWIGYIYPYNDGTKYNEKFKDKATLTSDKSSTTAYMDLSSLASEDSAVYYCARGGYYTYDDWGQGTTLTVSSGGGGSGGGGSGGGGSDVVMTQSPLSLPVSLGDQASISCRSSQSLVHSNGKTYLHWYLQKPGQSPKLLIYKVSNRFSGVPDRFSGSGSVTDFTLMISRVEAEDLGVYFCSQSTHVPYTFGGGTKLEIKDYKDDDDK

SEQ ID NO: 33 - DNA, 819 bp, Mus sp.
  CDS (1)..(813)
atgggatggagctgtatcatcctcttcttggtagcaacagctacaggtgtcgactcccaggtccagctgcagcagtctggacctgaactggtaaagcctggggcttcagtgaagatgtcctgcaaggcttctggatacaccttcgctaaccatgttattcactgggtgaagcagaagccagggcagggccttgagtggattggatatatttatccttacaatgatggtactaagtataatgagaagttcaaggacaaggccactctgacttcagacaaatcctccaccacagcctacatggacctcagcagcctggcctctgaggactctgcggtctattactgtgcaagagggggttactatacttacgacgactggggccaaggcaccactctcacagtctcctcaggtggtggtggttcgggtggtggtggttcgggtggtggcggatcggatgttgtgatgacccaaagtccactctccctgcctgtcagtcttggagatcaagcctccatctcttgcagatcaagtcagagccttgtgcacagtaatggaaagacctatttacattggtacctgcagaagccaggccagtctccaaaactcctgatctacaaagtttccaaccgattttctggggtcccagacaggttcagtggcagtggatcagtgacagatttcacactcatgatcagcagagtggaggctgaggatctgggagtttatttctgctctcaaagtacacatgttccgtacacgttcggaggggggaccaagctggaaataaaagactacaaagacgatgacgataaataatga

SEQ ID NO: 34 - protein, 271 aa, Mus sp.
MGWSCIILFLVATATGVDSQVQLQQSGPELVKPGASVKMSCKASGYTFANHVIHWVKQKPGQGLEWIGYIYPYNDGTKYNEKFKDKATLTSDKSSTTAYMDLSSLASEDSAVYYCARGGYYTYDDWGQGTTLTVSSGGGGSGGGGSGGGGSDVVMTQSPLSLPVSLGDQASISCRSSQSLVHSNGKTYLHWYLQKPGQSPKLLIYKVSNRFSGVPDRFSGSGSVTDFTLMISRVEAEDLGVYFCSQSTHVPYTFGGGTKLEIKDYKDDDDK

SEQ ID NO: 35 - DNA, 456 bp, Mus sp.
  CDS (1)..(450)
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatgactacaaggacgacgatgacaagtgatag

SEQ ID NO: 36 - protein, 150 aa, Mus sp.
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENDYKDDDDK

SEQ ID NO: 37 - DNA, 46 bp, Artificial Sequence
  Description of Artificial Sequence PCR Primer
ggaattccatatgcaagtgcaacttcaacagtctggacctgaactg

SEQ ID NO: 38 - DNA, 31 bp, Artificial Sequence
  Description of Artificial Sequence PCR Primer
ggaattctcattattttatttccagcttggt

SEQ ID NO: 39 - DNA, 741 bp, Mus sp.
  CDS (1)..(735)
atgcaagtgcaacttcaacagtctggacctgaactggtaaagcctggggcttcagtgaagatgtcctgcaaggcttctggatacaccttcgctaaccatgttattcactgggtgaagcagaagccagggcagggccttgagtggattggatatatttatccttacaatgatggtactaagtataatgagaagttcaaggacaaggccactctgacttcagacaaatcctccaccacagcctacatggacctcagcagcctggcctctgaggactctgcggtctattactgtgcaagagggggttactatacttacgacgactggggccaaggcaccactctcacagtctcctcaggtggtggtggttcgggtggtggtggttcgggtggtggcggatcggatgttgtgatgacccaaagtccactctccctgcctgtcagtcttggagatcaagcctccatctcttgcagatcaagtcagagccttgtgcacagtaatggaaagacctatttacattggtacctgcagaagccaggccagtctccaaaactcctgatctacaaagtttccaaccgattttctggggtcccagacaggttcagtggcagtggatcagtgacagatttcacactcatgatcagcagagtggaggctgaggatctgggagtttatttctgctctcaaagtacacatgttccgtacacgttcggaggggggaccaagctggaaataaaataatga

SEQ ID NO: 40 - protein, 245 aa, Mus sp.
MQVQLQQSGPELVKPGASVKMSCKASGYTFANHVIHWVKQKPGQGLEWIGYIYPYNDGTKYNEKFKDKATLTSDKSSTTAYMDLSSLASEDSAVYYCARGGYYTYDDWGQGTTLTVSSGGGGSGGGGSGGGGSDVVMTQSPLSLPVSLGDQASISCRSSQSLVHSNGKTYLHWYLQKPGQSPKLLIYKVSNRFSGVPDRFSGSGSVTDFTLMISRVEAEDLGVYFCSQSTHVPYTFGGGTKLEIK

(19) United States
(12) Patent Application Publication
(10) Pub. No.: US 2003/0108546 A1
(43) Pub. Date: Jun. 12, 2003

(54) Apoptosis-inducing single-chain Fv

(75) Inventors: Naoshi Fukushima, Gotemba-shi (JP); Shinsuke Uno, Gotemba-shi (JP); Masayoshi Oh-Eda, Gotemba-shi (JP); Yasufumi Kikuchi, Gotemba-shi (JP)

Correspondence Address: FOLEY AND LARDNER, SUITE 500, 3000 K STREET NW, WASHINGTON, DC, 20007, US

(73) Assignee: Chugai Seiyaku kabushiki kaisha

(21) Appl. No.: 10/138,505

(22) Filed: May 6, 2002

Related U.S. Application Data
(63) Continuation of application No. 09/523,095, filed on Mar. 10, 2000, now abandoned.

(30) Foreign Application Priority Data
Mar. 10, 1999 (JP) 11-63557

(51) Int. Cl. A61K 39/395; C07K 16/30; C07K 16/28

(52) U.S. Cl. 424/144.1; 424/155.1; 530/388.22; 530/388.8

(57) ABSTRACT

This invention relates to Novel single-chain Fvs capable of inducing apoptosis of nucleated blood cells having Integrin Associated Protein (IAP). The single-chain Fvs of the invention comprise an L chain comprising the L chain V region of the mouse monoclonal antibodies capable of inducing apoptosis of cells having human IAP, an H chain comprising the H chain V region of the mouse monoclonal antibodies capable of inducing apoptosis of cells having human IAP and a linker connecting them. The single-chain Fvs of the invention are useful as a therapeutic agent for blood dyscrasia such as leukemia.

### TECHNICAL FIELD

This invention relates to novel single-chain Fvs capable of inducing apoptosis of nucleated blood cells having Integrin Associated Protein (IAP). The single-chain Fvs are useful as a therapeutic agent for blood dyscrasia such as leukemia as described later.

### BACKGROUND ART

Granulocyte colony stimulating factors, such as recombinant granulocyte colony stimulating factor (rG-CSF), have been known in the prior art as humoral factors that stimulate differentiation and proliferation of granulocytes. Reports based on in vivo experiments with mice have shown that administration of rG-CSF results in not only accelerated myelopoiesis in bone marrow but also notable extramedullary hemopoiesis in the spleen, and hence proliferation of all hemopoietic precursor cells, including hemopoietic stem cells, in the spleen. The mechanism of such extramedullary hemopoiesis in the spleen has been believed that stimulation by rG-CSF alters the hemopoietic microenvironment of the spleen and promotes the hemopoiesis supporting ability thereof, thus inducing hemopoiesis.

In order to elucidate the hemopoietic function in the spleen, the present inventors focused on stromal cells of the spleen following repeated administration of rG-CSF. The inventors have made efforts to examine how the hemopoietic function is promoted by rG-CSF via stromal cells, and have established a hemopoietic stromal cell line (CF-1 cells) from mouse spleen by repeated administration of rG-CSF. The inventors have studied the hemopoiesis supporting ability of the hemopoietic stromal cells and confirmed the colony stimulating activity in vitro and the hemopoietic stem cell supporting ability in vivo [Blood, 80, 1914 (1992)].

However, one cell line of the splenic stromal cells has been established (CF-1 cells) and its cytological characteristics have been studied, whereas specific antibodies which recognize the surface antigens of these cells have never been prepared, nor have their characteristics been elucidated yet in any way.

In light of the aforementioned findings relating to splenic stromal cells and the results of prior research, the present inventors have earnestly made further research aiming at developing specific antibodies that can recognize the splenic stromal cells, made efforts to prepare monoclonal antibodies using the aforementioned splenic stromal cell line as a sensitizing antigen, and finally succeeded in obtaining novel monoclonal antibodies.

The inventors have further studied identities of the monoclonal antibodies obtained as above and found that the monoclonal antibodies are capable of inducing apoptosis of myeloid cells.

The inventors have also examined an antigen recognized by the antibody and found that the antigen is mouse Integrin Associated Protein (mouse IAP) (GeneBank, Accession Number Z25524).

The action of the antibodies has been studied using recombinant cells in which the mouse IAP gene had been introduced (Japanese Patent Application No. 09-67499).

In light of the aforementioned findings, the present inventors have succeeded in obtaining monoclonal antibodies of which the antigen is human Integrin Associated Protein (hereinafter referred to as human IAP; amino acid sequence and nucleotide sequence thereof are described in J. Cell Biol., 123, 485-496, 1993; see-also Journal of Cell Science, 108, 3419-3425, 1995) and which are capable of inducing apoptosis of human nucleated blood cells having said antigen.

Further, the present inventors have succeeded in obtaining hybridomas which can produce novel monoclonal antibodies capable of inducing apoptosis of nucleated blood cells (myeloid cells and lymphocytes) having human Integrin Associated Protein (human IAP).

These hybridomas are hereinafter referred to MABL-1 (FERM BP-6100) and MABL-2 (FERM BP-6101), and monoclonal antibodies produced by the hybridomas are also referred to MABL-1 antibody and MABL-2 antibody, respectively.

### DISCLOSURE OF INVENTION

The inventors have earnestly studied to utilize the aforementioned monoclonal antibodies derived from the mice and having human IAP as antigen as a therapeutic agent for the after-mentioned blood dyscrasia.

An object of this invention is to provide antibodies which include a novel single-chain Fv capable of inducing apoptosis of nucleated blood cells having human IAP. In the invention, the term “a single-chain Fv” means a single chain polypeptide comprising an H chain V region and an L chain V region of the monoclonal antibodies.

Another object of the present invention is to provide therapeutic agents for blood dyscrasia comprising the substance obtained as above which is capable of inducing apoptosis of nucleated blood cells having Integrin Associated Protein (IAP).

The present invention relates to single chain antibodies obtainable by reconstruction of the monoclonal antibodies derived from mice. Specifically, the invention relates to the reconstructed single-chain Fvs obtainable from the mouse monoclonal antibodies which are capable of inducing apoptosis of nucleated blood cells having human IAP.

The present invention also relates to humanized antibodies of the reconstructed single-chain Fvs. Further, the invention relates to humanized monoclonal antibodies and fragments thereof which are producible from the foregoing humanized antibodies by the gene engineering approaches. The invention further provides human/mouse chimera antibodies, which are obtainable in the course of the production of the reconstructed single-chain Fvs.

The present invention further relates to the process for genetically producing the reconstructed single-chain Fv of the mouse monoclonal antibodies, the humanized reconstructed single-chain Fv, the humanized monoclonal antibodies and fragments thereof and the chimera antibodies.

Specifically, the present invention relates to single-chain Fvs capable of inducing apoptosis of nucleated blood cells having human IAP, which comprise the L chain V region and the H chain V region of the mouse monoclonal antibodies (MABL-1 and MABL-2 antibodies) capable of inducing apoptosis of nucleated blood cells having human IAP. The invention further relates to single-chain Fvs, wherein amino acid sequences of these V regions are partially modified.

Additionally, the present invention relates to the reconstructed humanized single-chain Fv, the reconstructed humanized monoclonal antibodies and the fragments of the humanized monoclonal antibodies, which are capable of inducing apoptosis of nucleated blood cells having human IAP, and which are constructed of the reconstructed humanized L chain V region comprising a framework region (FR) and a CDR of the aforementioned mouse monoclonal antibodies, and the reconstructed humanized H chain V region comprising an FR and a CDR of the aforementioned mouse monoclonal antibodies. The invention also relates to the reconstructed humanized single-chain Fvs, the reconstructed humanized monoclonal antibodies and fragments thereof which have the same effect and in which amino acid sequences are partially modified.

Furthermore, the present invention relates to chimera antibodies capable of inducing apoptosis of nucleated blood cells having human IAP, which comprise an L chain comprising an L chain C region of human antibodies and an L chain V region of the aforementioned mouse monoclonal antibodies, and an H chain comprising an H chain C region of human antibodies and an H chain V region of the aforementioned mouse monoclonal antibodies.

The invention also relates to DNAs encoding the aforementioned antibodies, recombinant vectors comprising the DNAs and hosts transformed with the recombinant vectors.

The invention relates to a process for producing the reconstructed single-chain Fvs and the modified single-chain Fvs in which amino acid sequences are partially modified, which comprises culturing the above hosts and extracting the reconstructed single-chain Fvs from the culture thereof.

The invention further relates to a process for the production of the reconstructed humanized single-chain Fvs, the reconstructed humanized monoclonal antibodies and fragments thereof, and the reconstructed humanized single-chain Fvs, the reconstructed humanized monoclonal antibodies and fragments thereof in which amino acid sequences are partially modified, which are capable of inducing apoptosis of nucleated blood cells having human IAP.

The invention further relates to a process for producing the chimera antibodies capable of inducing apoptosis of nucleated blood cells having human IAP.

The present invention relates to therapeutic agents for blood dyscrasia comprising the substance as obtained above which is capable of inducing apoptosis of nucleated blood cells having Integrin Associated Protein (IAP).

There is no method for producing the reconstructed single-chain Fvs, which is applicable to the production of any specific antibodies and thus various means are needed to produce a reconstructed single-chain Fv sufficiently active to a specific antigen. Generally, a single chain antibody can be formed from a monoclonal antibody in the following manner. That is, it can be attained by linking the H chain V region and the L chain V region derived from the monoclonal antibodies by using a linker. The resulting reconstructed single-chain Fvs contain variable regions of the parent antibodies and the complementarity determining region (CDR) thereof are preserved, and therefore the single-chain Fvs can be expected to bind to the antigen by the same specificity as that of the parent monoclonal antibodies.

In the present invention, the processes for producing the reconstructed single-chain Fvs as aforementioned are employed.

Cloning of a DNA Encoding V Region of Mouse Antibodies

In order to isolate a DNA encoding the V region of the mouse monoclonal antibodies for human IAP, mRNAs are prepared from cells producing the mouse monoclonal antibody and converted to double strand cDNAs by the conventional method and the desired DNA is amplified from the cDNAs by polymerase chain reaction (PCR) method. As a source of mRNAs, the preparation of a hybridoma producing a monoclonal antibody to human IAP is required. MABL-1 (FERM BP-6100) or MABL-2 (FERM BP-6101) can be exemplified as the hybridoma. The monoclonal antibodies produced by the hybridomas, MABL-1 and MABL-2, are hereinafter referred to MABL-1 antibody and MABL-2 antibody, respectively. A process for producing the hybridoma MABL-1 or MABL-2 will be hereinafter described in Referential Example 1.

(1) Extraction of Total RNA

In the present invention, hybridoma cells are lysed with ISOGEN (Nippon Gene Inc.) and the resultant lysate is treated with isopropanol in order to extract total RNA. The processes which have already been used for the cloning of a gene of other protein can also be employed, for example, the process using the treatment with guanidine isothiocyanate followed by density-gradient centrifugation by cesium chloride (Chirgwin, J. M. et al., Biochemistry, 18, 5294-5299, 1979) and the process using the treatment with a surfactant in the presence of a ribonuclease inhibitor such as a vanadium complex followed by phenol treatment (Berger, S. L. et al., Biochemistry, 18, 5143-5149, 1979).

(2) Preparation of Double-Strand cDNA

For the preparation of single strand DNAs from the total RNA prepared as above, the total RNA as a template is treated with a reverse transcriptase using oligo(dT) as a primer which is complementary to the poly A chain located at 3′-end of the RNA and the single strand DNA (cDNA) complementary to the total RNA can be synthesized (Larrik, J. W. et al., Bio/Technology, 7, 934-938, 1989). At that time, random primers may also be used.

(3) Amplification of V Region of Mouse Antibody by Polymerase Chain Reaction (PCR)

The V region of the mouse antibody is specifically amplified from the cDNAs using the polymerase chain reaction (PCR). For the amplification of the V region of the mouse antibody, primers described in Jones, S. T. et al., Bio/Technology, 9, 88-89, 1991 may be employed. In order to select primers to be used for cloning the mouse monoclonal antibody produced by the hybridoma, MABL-1 or MABL-2, the typing of both H and L chains should be carried out.

The typing using ITOTYPING KIT (STRATAGENE Inc.) reveals that the MABL-1 antibody has a κ type L chain and a γ1 type H chain and that the MABL-2 antibody has a κ type L 10-chain and a γ2a type H chain. The typing will be described in Referential Example 2.

Oligonucleotide primers of SEQ ID No. 1 and SEQ ID No. 2 are used as 5′-end and 3′-end primers, respectively, in order to amplify the L chain V region of the MABL-1 antibody by means of the polymerase chain reaction (PCR). The oligonucleotide primers of SEQ ID No. 1 and SEQ ID No. 2 are used as 5′-end and 3-end primers, respectively, in order to amplify the L chain V region of the MABL-2 antibody.

The oligonucleotide primers of of SEQ ID No. 1 and SEQ ID No. 3 are used as 5′-end and 3′-end primers, respectively, in order to amplify the H chain V region of the MABL-1 antibody. The oligonucleotide primers of SEQ ID No. 1 and SEQ ID No. 4 are used as 5′-end and 3′-end primers, respectively, in order to amplify the H chain V region of the MABL-2 antibody.

In embodiments of the invention, the 5′-primers which contain a sequence “GANTC” providing the restriction enzyme Hinf I digestion site at the neighborhood of 5′-terminal thereof are used and the 3′-primers which contain a nucleotide sequence “CCCGGG” providing the XmaI digestion site at the neighborhood of 5′-terminal thereof are used. Other restriction enzyme digestion sites may be used instead of these sites as long as they are used for subcloning a desired DNA fragment into a cloning vector.

The amplified product is isolated and purified using a low-melting temperature agarose or a column (PCR products purification kit (e.g. QIAGEN) or a DNA purification kit (e.g. GENECLEAN II) to obtain a desired DNA fragment encoding the variable region. A plasmid containing the DNA fragment encoding the desired variable region of the mouse monoclonal antibody is obtainable by linking the DNA fragment to a suitable cloning vector such as pGEM-T Easy.

Sequencing of cloned DNAs can be carried out by any conventional method, for example, an automatic DNA sequencer (Applied Biosystems Inc.).

The cloning and the sequencing of the desired DNAs will concretely be described in Examples 1 and 2.

Complementarity Determining Region (CDR)

Each pair of the V regions of L and H chain forms an antigen binding site. The variable regions of the L and H chains link to comparatively conserved four framework regions with commonality and three hypervariable regions or complementarity determining regions (CDR) (Kabat, E. A. et al., “Sequences of Protein of Immunological Interest”, US Dept. Health and Human Services, 1983).

Major portions in the four framework regions (FRS) form β-sheet structures and thus three CDRs form a loop. CDRs may form a part of the β-sheet structure in a certain case. The three CDRs are mutually held at the structurally closed position and contribute to the formation of the antigen binding site together with the three CDRs in the region forming a pair.

These CDRs can be found out by comparing the amino acid sequence of V region of the obtained antibody with known amino acid sequences of V regions of known antibodies according to the empirical rule in Kabat, E. A. et al., “Sequences of Protein of Immunological Interest”. This will concretely be illustrated in Example 3.

Preparation of Chimera Antibody

Prior to designing a single-chain Fv reconstructed from an antibody for human IAP, it should be confirmed that the employed CDRs actually form an antigen binding site. For this purpose, a chimera antigen is prepared. The amino acid sequences which are assumed from nucleotide sequences of the cloned DNAs of the monoclonal MABL-1 and MABL-2 antibodies described in Example 1 are compared with an amino acid sequence of the V region of the known mouse monoclonal antibody.

Cloning of a DNA fragment encoding V regions of L and H chains of the monoclonal antibody allows to prepare a chimera MABL-1 antibody or a chimera MABL-2 antibody by linking the resultant DNA encoding the mouse V region to a DNA encoding constant region of human antibody.

A basic method for preparing a chimera antibody comprises linking a mouse leader sequence and a sequence of V region existing in the cloned cDNA to a sequence coding the C region of a human antibody existing in an expression vector for mammal cells. The C region of the aforementioned human antibody may be any one of human L chain C regions and human H chain C regions, for example, human L chain Cκ, H chain γ-1 C and γ-4 C regions.

For the preparation of the chimera antibody, two expression vectors are prepared; i.e., an expression vector comprising a DNA encoding the mouse L chain V region and the human L chain C region under the control of an expression regulation region such as an enhancer/promoter system, and an expression vector comprising a DNA encoding the mouse H chain V region and the human H chain C region under the control of an expression regulation region such as an enhancer/promoter system. Then, a host cell such as a mammalian cell is co-transformed with these expression vectors and the transformed cell is cultured in vitro or in vivo to prepare the chimera antibody (e.g. WO91-16928).

Alternatively, a DNA encoding the mouse L chain V region and the human L chain C region and a DNA encoding the mouse H chain V region and the human H chain C region are introduced into a single expression vector, a host cell is transformed with the vector and the transformed host is cultured in vitro or in vivo in order to produce the desired chimera antibody.

The preparation of the chimera antibody will be described in Example 4.

A CDNA encoding a leader region and a V region of the L chain of the MABL-1 or MABL-2 antibody is subcloned by PCR method and linked to an expression vector containing a genome DNA encoding a human genomic L chain C region.

A cDNA encoding an H chain leader region and a V region of the γ1 type of MABL-1 or MABL-2 antibody is subcloned by PCR method and linked to an expression vector containing a genome DNA encoding a human genomic L chain Cκ region.

Specifically designed PCR primers are employed to provide suitable nucleotide sequences at 5′-end and 3′-end of the CDNAs encoding the V regions of the MABL-1 and MABL-2 antibodies so that the cDNAs are readily inserted into an expression vector and appropriately function in the expression vector (e.g. this invention devises to increase transcription efficiency by inserting Kozak sequence). The V regions of the MABL-1 and MABL-2 antibodies obtained by amplifying by PCR using these primers are inserted into HEF expression vector containing the desired human C region (see WO92-19759). The vector is suitable for a transient expression or a stable expression of genetically modified antibodies in various mammalian cell lines.

The chimera MABL-1 and MABL-2 antibodies demonstrate an activity to bind to cells having human IAP. This confirms that correct mouse V regions have been cloned and that their sequences have been determined.

Reconstructed Single-Chain Fv

For the production of a reconstructed single-chain Fv for cells having human IAP, the H chain V region and the L chain V region of the monoclonal antibody to human IAP are connected via a linker, preferably a peptide linker. A peptide linker includes any single chain linkers comprising 12 to 19 amino acids, for example, a peptide fragment described in SEQ ID No. 19.

Concrete amino acid sequences of the reconstructed single-chain Fv are exemplified in SEQ ID Nos. 20, 23, 24 and 25. In the invention, the single-chain Fvs having the amino acid sequences are referred to as MABL1-scFV and MABL2-scFv, which will be illustrated in Example 5.

The reconstructed single-chain Fvs of the invention are obtainable in the following manner; the DNA encoding the H chain V region of the MABL-1 or MABL-2 antibody and the DNA encoding the L chain V region of the MABL-1 or MABL-2 antibody, which are illustrated hereinabove, are employed as templates and a DNA encoding the desired amino acid sequence within these sequences is amplified by PCR method using a pair of primers which define both ends thereof.

A process for producing the reconstructed single-chain Fv comprising the H chain V region and the L chain V region will be concretely described in Example 5.

The antigen-binding activity of the reconstructed single-chain Fv can be evaluated in terms of the binding-inhibitory ability of the mouse MABL-1 and MABL-2 antibodies to human IAP as an index. Actually, the concentration dependent inhibition of the mouse MABL-2 antibody to human IAP antigen is observed.

Preferably, an amino acid sequence of the aforementioned V regions may partially be modified in order to produce a reconstructed single-chain Fv which is sufficiently active for a specific antigen, if necessary.

The reconstructed single-chain Fv according to the present invention can be humanized by using conventional techniques (e.g. Sato, K. et al., Cancer Res., 53, 1-6 (1993)). Once a DNA encoding a humanized Fv is prepared, a humanized single-chain Fv, a fragment of the humanized single-chain Fv, a humanized monoclonal antibody and a fragment of the humanized monoclonal antibody can readily be produced according to conventional methods. Preferably, amino acid sequences of the V regions thereof are partially modified, if necessary.

As mentioned above, when the objective DNAs encoding the reconstructed single-chain Fv, the reconstructed humanized single-chain Fv, the humanized monoclonal antibodies and fragments thereof are prepared, the expression vectors containing them and hosts transformed with the vectors can be obtained according to conventional methods. Further, the hosts can be cultured according to a conventional method to produce the reconstructed single-chain Fv, the reconstructed humanized single-chain Fv, the humanized monoclonal antibodies and fragments thereof. These can be isolated from cells or a medium and can be purified uniformly, for which any isolation and purification method conventionally used for proteins may be employed without limitation thereto. The chimera antibodies or the humanized antibodies can be isolated and purified by suitable selection or combination of the methods, for example, various chromatographs, ultrafiltration, salting-out and dialysis.

For the production of the reconstructed single-chain Fv, the humanized single-chain Fv and the humanized monoclonal antibodies and fragments thereof against cells having human IAP according to the present invention, any expression systems can be employed, for example, eukaryotic cells such as an animal cell, e.g., an established mammalian cell line, filamentous fungi and yeast, and prokaryotic cells such as a bacterial cell, e.g., E. coli. Preferably, the chimera antibody or the reconstructed antibody of the invention is expressed in a mammal cell, for example COS7 cell or CHO cell.

In these cases, conventional promoters useful for the expression in a mammal cell can be used. Preferably, human cytomegalovirus (HCMV) immediate early promoter can be used. Expression vectors containing the HCMV promoter include HCMV-VH-HCγ 1, HCMV-VL-HCK and the like which are derived from pSV2neo (WO92-19759).

Additionally, other promoters for gene expression in mammal cell which may be used in the invention include virus promoters derived form retrovirus, polyoma virus, adenovirus and simian virus 40 (SV40) and promoters derived from mammal such as human polypeptide-chain elongation factor-1α (HEF-1α). SV40 promoter can easily be used according to the method of Mulligan, R. C., et al. (Nature 277, 108-114 (1979)) and HEF-1α promoter can also be used according to the methods of Mizushima, S. et al. (Nucleic Acids Research, 18, 5322 (1990)).

Replication origin (ori) which can be used in the invention includes ori derived from SV40, polyoma virus, adenovirus, bovine papilloma virus (BPV) and the like. For the purpose of the amplification of gene copy number in the host cell system and the like, an expression vector may contain phosphotransferase APH (3′) II or I (neo) gene, thymidine kinase (TK) gene, E. coli xanthine-guanine phosphoribosyl transferase (Ecogpt) gene or dihydrofolate reductase (DHFR) gene.

The binding activity of the reconstructed polypeptide as prepared above to the antigen can be evaluated using the binding-inhibitory ability of the mouse MABL-1 and MABL-2 antibodies to human IAP as an index. Concretely, the activity is evaluated in terms of the absence or presence of concentration dependent inhibition of the binding of the mouse MABL-2 antibody to human IAP antigen as an index.

Actually, animal cells transformed with an expression vector containing a DNA encoding the reconstructed polypeptide of the invention, e.g., COS7 cell or CHO cell, are cultured and the cultured cells and/or the cultured supernatant or the reconstructed polypeptide purified from them are used to determine the binding to antigen. As a control, the cultured supernatant from cells transformed with the expression vector only is used. A test sample of the reconstructed polypeptide of the invention or the cultured supernatant of control is added to mouse leukemia cell line, L1210 cell, expressing human Integrin Associated Protein (IAP) and then an assay such as the flow cytometry is carried out to evaluate the binding activity to antigen.

The effect of inducing apoptosis in vitro is evaluated in the following manner: A test sample of the above reconstructed polypeptide is added to the cells into which the human IAP gene has been introduced and the sample is evaluated on its inducibility of human IAP-specific cell death in the cells.

The effect of inducing apoptosis in vivo is evaluated in the following manner: A model mouse of human myeloma is prepared. To the mouse is intravenously administered the monoclonal antibody or the reconstructed polypeptide of the invention, which is capable of inducing apoptosis of nucleated blood cells having IAP. To mice of a control group is administered PBS alone. The induction of apoptosis is evaluated in terms of antitumor effect by the change of human IgG content in serum of the mice and the survival time.

Hemagglutinating effect is tested in the following manner: Erythrocyte suspending fluid is prepared from blood collected from healthy men. Test samples of different concentrations are added to the fluid, which are then incubated to determine the hemagglutination.

Polypeptide of the invention, which contains two H chain V regions and two L chain V regions, is a dimer of single-chain Fv comprising an H chain V region and an L chain V region or a polypeptide monomer linking two H chain V regions and two L chain V regions. It is considered that the peptide of the aforementioned construction mimics the three dimensional structure of the antigen binding site of the parent monoclonal antibody and therefore has an excellent antigen-binding property.

The polypeptide of the invention has a superior mobility to tissues or tumors over whole IgG and a remarkably reduced or no side effect of hemagglutination. Therefore, it is expected that the peptide of the invention can be used as a therapeutic agent for blood dyscrasia, for example, leukemia such as acute myeloid leukemia, chronic myelogenous leukemia, acute lymphoblastic leukemia, chronic lymphoblastic leukemia, adult T-cell leukemia, multiple myeloma, mixed leukemia and hairy cell leukemia, malignant lymphoma (Hodgkin's disease, non-Hodgkin's lymphoma), hypoplastic anemia, osteomyelodysplasia and polycythemia vera. It is further expected that the peptide of the invention can be used as a contrast agent by RI-labeling and the effect of the peptide can be enhanced by attaching to a RI-compound or a toxin.

### EXPLANATION OF DRAWINGS

FIG. 1 shows the result of flow cytometry, illustrating that human IgG antibody does not bind to L1210 cells expressing human IAP (hIAP/L1210).

FIG. 2 shows the result of flow cytometry, illustrating that the chimera MABL-1 antibody specifically binds to L1210 cells expressing human IAP (hIAP/L1210).

FIG. 3 shows the result of flow cytometry, illustrating that the chimera MABL-2 antibody specifically binds to L1210 cells expressing human IAP (hIAP/L1210).

FIG. 4 schematically illustrates the process for producing the single-chain Fv according to the present invention.

FIG. 5 illustrates a structure of an expression plasmid which can be used to express a DNA encoding the single-chain Fv of the invention in E. coli.

FIG. 6 illustrates a structure of the expression plasmid which is used to express a DNA encoding the single-chain Fv of the invention in mammal cells.

FIG. 7 shows a photograph showing the result of western blotting in Example 5.4. From the left, a molecular weight marker (which indicates 97.4, 66, 45, 31, 21.5 and 14.5 kDa from the top), the cultured supernatant of pCHO1-introduced COS7 cells and the cultured supernatant of pCHOM2-introduced COS7 cells. The figure shows that the reconstructed single-chain Fv of the MABL-2 antibody (arrow) is contained in the cultured supernatant of the pCHOM2-introduced cells.

FIG. 8 shows the result of flow cytometry, illustrating that an antibody in the cultured supernatant of pCHO1/COS7 cell as a control does not bind to pCOS1/L1210 cell as a control.

FIG. 9 shows the result of flow cytometry, which illustrates that an antibody in the cultured supernatant of MABL2-scFv/COS7 cells does not bind to pCOS1/L1210 cells as a control.

FIG. 10 shows the result of flow cytometry, illustrating that an antibody in the cultured supernatant of pCHO/COS7 cells as a control does not bind to hIAP/L1210 cells.

FIG. 11 shows the result of flow cytometry, illustrating that an antibody in the cultured supernatant of MABL2-scFv/COS7 cells specifically binds to hIAP/L1210 cells.

FIG. 12 shows the result of the competitive ELISA in Example 5.6, wherein the binding activity of the single-chain Fv of the invention (MABL2-scFv) to the antigen is demonstrated in terms of the inhibition of binding of the mouse monoclonal antibody MABL-2 to the antigen as an index, in comparison with the cultured supernatant of pCHO1/COS7 cells as a control.

FIG. 13 shows the results of the apoptosis induction in Example 5.7, illustrating that the antibody in the cultured supernatant of pCHO1/COS7 cells as a control does not induce the apoptosis of pCOS1/L1210 cells as a control.

FIG. 14 shows the results of the apoptosis induction in Example 5.7, illustrating that the antibody in the cultured supernatant of MABL2-scFv/COS7 cells does not induce apoptosis of pCOS1/L1210 cells as a control.

FIG. 15 shows the results of the apoptosis induction in Example 5.7, illustrating that the antibody in the cultured supernatant of pCHO1/COS7 cells as a control does not induce apoptosis of hIAP/L1210 cells.

FIG. 16 shows the results of the apoptosis induction in Example 5.7, illustrating that the antibody in the cultured supernatant of MABL2-scFv/COS7 cells specifically induces apoptosis of hIAP/L1210 cells.

FIG. 17 shows the results of the apoptosis induction in Example 5.7, illustrating that the antibody in the cultured supernatant of pCHO1/COS7 cells as a control does not induce apoptosis of CCRF-CEM cells (at 50% of the final concentration).

FIG. 18 shows the results of the apoptosis induction in Example 5.7, illustrating that the antibody in the cultured supernatant of MABL2-scFv/COS7 cells specifically induces apoptosis of CCRF-CEM cells (at 50% of the final concentration).

FIG. 19 shows the chromatogram of the purification using hydroxyapatite column of the fractions from Blue-sepharose column in the course of purification of the single-chain Fv derived form the MABL-2 antibody produced by the CHO cells in Example 5.9, wherein fractions A and B are obtained as the major peaks.

FIG. 20 shows the results of purification of fractions A and B obtained in Example 5.9-(2), wherein the major peaks (AI and BI, respectively) are eluted at approximately 36 kD of the apparent molecular weight from fraction A and at approximately 76 kD of the apparent molecular weight from fraction B.

FIG. 21 is the analysis on SDS-PAGE of the fractions obtained in the course of purification of the single chain Fv derived form the MABL-2 antibody produced by the CHO cells in Example 5.9, showing that a single band of approximately 35 kD of molecular weight is observed in both fractions.

FIG. 22 shows the results of analysis of fractions AI and BI obtained by gel filtration in the course of purification of the single-chain Fv derived form the MABL-2 antibody, wherein fraction AI comprises monomer and fraction BI comprises dimer.

FIG. 23 illustrates a structure of an expression plasmid which can be used to express a DNA encoding the single-chain Fv of the invention in E. coli.

FIG. 24 shows the results of purification on the gel filtration column of crude products obtained in the course of purification of the single-chain Fv polypeptide derived form the MABL-2 antibody produced by E. coli in Example 5.12, each peak indicating monomer or dimer of the single-chain Fv produced by E. coli.

FIG. 25 shows the results of the apoptosis induction in Example 5.13, showing that mouse IgG antibody as a control does not induce apoptosis of hIAP/L1210 cells (at the final concentration of 3 μg/ml).

FIG. 26 shows the results of the apoptosis induction in Example 5.13, showing that the dimer of MABL2-scFv produced by the CHO cells remarkably induces apoptosis of hIAP/L1210 cells (at the final concentration of 3 μg/ml).

FIG. 27 shows the results of the apoptosis induction in Example 5.13, showing that the dimer of MABL2-scFv produced by E. coli remarkably induces apoptosis of hIAP/L1210 cells (at the final concentration of 3 μg/ml).

FIG. 28 shows the results of the apoptosis induction in Example 5.13, showing that, for hIAP/L1210 cells, apoptosis-inducing action of the MABL2-scFv monomer produced by the CHO cells is nearly equal to that of the control (at the final concentration of 3 μg/ml).

FIG. 29 shows the results of the apoptosis induction in Example 5.13, showing that apoptosis-inducing action of the MABL2-scFv monomer produced by E. coli is nearly equal to that of control (at the final concentration of 3 μg/ml).

FIG. 30 shows the results of quantitative measurement of human IgG in the serum of a human myeloma cell line KPMM2-transplanted mouse, indicating amounts of human IgG produced by the human myeloma in the mouse. The figure shows that the dimer of scFv/CHO remarkably inhibits growth of the KPMM2 cells.

FIG. 31 shows the survival time of the mouse after the transplantation, illustrating the remarkably elongated survival time in the scFv/CHO dimer-administered group.

The present invention will concretely be illustrated in reference to the following examples, which in no way limit the scope of the invention.

### EXAMPLE

### Referential Example 1

### Preparation of Hybridoma

The cells highly expressing human Integrin Associated Protein (IAP) in L1210 cells, which are Leukemia cell line derived from DBA mouse (ATCC No. CCL-219; J. Natl. Cancer Inst. 10: 179-192, 1949), was prepared as described below and the cells were used as a sensitizing antibody.

The human IAP gene was amplified by PCR using cDNA prepared from mRNA of HL-60 cell line (CLONETECH Inc.,) as a template.

This PCR product was introduced into a cloning vector, pGEM-T vector (Promega Inc.,) and E. coli, JM109 (Takara Inc.), was transformed with the resulting vector. A nucleotide sequence of the insert DNA was confirmed using a DNA sequencer (373 DNA Sequencer, ABI Inc.,) and then the insert DNA was recombined with an expression vector, pCOS1.

The expression vector pCOS1, which is a derivative of PEF-BOS (Nucleic Acids Research, 18, 5322, 1990), employs human elongation factor-la as a promoter/enhancer and incorporates the neomycin resistant gene. This expression vector with human IAP incorporated was transfected to L1210 cell line with DMRIE-C (GIBCO-BRL). The L1210 cells were selected using Geneticin (final concentration: 1 mg/ml, GIBCO-BRL) and cloned by the limiting dilution method. For the resulting clones, an expression of the antigen, human IAP, was analyzed using the anti-CD47 antibody recognizing human IAP (PharMingen) and a clone highly expressing the antigen was selected as an antigen-sensitizing cell.

Cell fusion between splenic cells of DBA/2 mouse (Japan Charles River Reproduction Inc.) which had been immunized with the aforementioned cells and mouse myeloma cell line P3-U1 (Current Topics in Micro-biology and Immunology, 81, 1-7 (1978)) was carried out according to a conventional method using polyethylene glycol (Clin. Exp. Immunol., 42, 458-462 (1980)). The DBA/2 mouse was the same strain as the L1210 cells.

Screening was performed using an activity of specifically recognizing human IAP as an indicator and two hybridomas were established. These have been designated as MABL-1 and MABL-2 and were internationally deposited as FERM BP-6100 and FERM BP-6101 on Sep. 11, 1997 with the National Institute of Bioscience and Human Technology, Agency of Industrial Science and Technology, Minister of International Trade and Indusry of 1-3 Higasi 1-chome, Tsukuba-shi, Ibaraki-ken, Japan, as an authorized depository for microorganisms.

### Referential Example 2

### Subclass Identification of MABL-1 and MABL-2 Antibodies

In order to identify the subclasses of the MABL-1 and MABL-2 antibodies as obtained above, 500 μl each of the MABL-1 and MABL-2 antibodies prepared at a level of 100 ng/ml was spotted on the Isotyping Kit (STRATAGENE).

Consequently, it was revealed that the MABL-1 antibody is IgGI, K type and the MABL-2 antibody is IgG2a, K type. 0.20

### Example 1

### Cloning of DNAs Encoding V Region of Mouse Monoclonal Antibodies to Human IAP

DNAs encoding variable regions of the mouse monoclonal antibodies, MABL-1 and MABL-2, to human IAP were cloned as follows.

1.1 Preparation of Messenger RNA (mRNA)

mRNAs were prepared from the hybridomas MABL-1 and MABL-2 using the mRNA Purification Kit (Pharmacia Biotech).

1.2 Synthesis of Double Strand CDNA

Double strand CDNA was synthesized from about 1 μg of the mRNA using Marathon CDNA Amplification Kit (CLONTECH) and an adapter was linked thereto.

1.3 Amplification of Genes Encoding Variable Regions of an Antibody by PCR

PCR was carried out using the Thermal Cycler (PERKIN ELMER).

(1) Amplification of a Gene Coding L Chain V Region of MABL-1

Primers used for the PCR method are Adapter Primer-1 (CLONTECH) shown in SEQ ID No. 1 which hybridizes to a partial sequence of the adapter and MKC (Mouse Kappa Constant) primer (Bio/Technology, 9, 88-89, 1991) shown in SEQ ID No. 2 which hybridizes to the mouse kappa type L chain V region.

50 μl of the PCR solution contains 5 μl of 10×PCR Buffer II, 2 mM MgCl₂, 0.16 mM dNTPs (DATP, dGTP, dCTP and dTTP), 2.5 units of a DNA polymerase, AmpliTaq Gold (PERKIN ELMER), 0.2 μM of the adapter primer of SEQ ID No. 1, 0.2 μM of the MKC of SEQ ID No. 2 and 0.1 μg of the double strand cDNA derived from MABL-1. The solution was preheated at 94° C. of the initial temperature for 9 minutes and then heated at 94° C. for 1 minute, at 60° C. for 1 minute and at 72° C. for 1 minute 20 seconds in this order. This temperature cycle was repeated 35 times and then the reaction mixture was further heated at 72° C. for 10 minutes.

(2) Amplification of cDNA Encoding H Chain V Region of MABL-1

The Adapter Primer-1 shown in SEQ ID No. 1 and MHC-γ1 (Mouse Heavy Constant) primer (BioTechnology, 9, 88-89, 1991) shown in SEQ ID No. 3 were used as primers for PCR.

The amplification of CDNA was performed according to the method of the amplification of the L chain V region gene, which was described in Example 1.3-(1), except for using 0.2 μM of the MHC-γ1 primer instead of 0.2 μM of the MKC primer.

(3) Amplification of cDNA Encoding L Chain V Region of MABL-2

The Adapter Primer-1 of SEQ ID No. 1 and the MKC primer of SEQ ID No. 2 were used as primers for PCR.

The amplification of cDNA was carried out according to the method of the amplification of the L chain V region gene of MABL-1 which was described in Example 1.3-(1), except for using 0.1 μg of the double strand cDNA derived from MABL-2 instead of 0.1 μg of the double strand cDNA from MABL-1.

(4) Amplification of cDNA Encoding H Chain V Region of MABL-2

The Adapter Primer-1 of SEQ ID No. 1 and MHC-γ2a primer (Bio/Technology, 9, 88-89, 1991) shown in SEQ ID No. 4 were used as primers for PCR.

The amplification of CDNA was performed according to the method of the amplification of the L chain V region gene, which was described in Example 1.3-(3), except for using 0.2 μM of the MHC-γ2a primer instead of 0.2 μM of the MKC primer.

1.4 Purification of PCR Products

The DNA fragment amplified by PCR as described above was purified using the QIAquick PCR Purification Kit (QIAGEN) and dissolved in 10 mM Tris-HCl (pH 8.0) containing 1 mM EDTA.

1.5 Ligation and Transformation

About 140 ng of the DNA fragment comprising the gene coding the mouse kappa type L chain V region derived from MABL-1 as prepared above was ligated with 50 ng of PGEM-T Easy vector (Promega) in the reaction buffer comprising 30 mM Tris-HCl (pH 7.8), 10 mM MgCl₂, 10 mM dithiothreitol, 1 mM ATP and 3 units of T4 DNA Ligase (Promega) at 15° C. for 3 hours.

Then, 1 μl of the reaction mixture was added to 50 μl of E. coli DH5a competent cells (Toyobo Inc.) and the cells were stored on ice for 30 minutes, incubated at 42° C. for 1 minute and stored on ice for 2 minutes again. 100 μl of SOC medium (GIBCO BRL) was added and the cells were plated on LB (Molecular Cloning: A Laboratory Manual, Sambrook et al., Cold Spring Harbor Laboratory Press, 1989) agar medium containing 100 μg/ml of ampicillin (SIGMA) and cultured at 37° C. overnight to obtain the transformant of E. coli.

The transformant was cultured in 3 ml of LB medium containing 50 μg/ml of ampicillin at 37° C. overnight and the plasmid DNA was prepared from the culture using the QIAprep Spin Miniprep Kit (QIAGEN).

The resulting plasmid comprising the gene coding the mouse kappa type L chain V region derived from the hybridoma MABL-1 was designated as pGEM-M L.

According to the same manner as described above, a plasmid comprising the gene coding the mouse H chain V region derived from the hybridoma MABL-1 was prepared from the purified DNA fragment and designated as pGEM-M1H.

A plasmid comprising the gene coding the mouse kappa type L chain V region derived from the hybridoma MABL-2 was prepared from the purified DNA fragment and designated as pGEM-M2L.

A plasmid comprising the gene coding the mouse H chain V region derived from the hybridoma MABL-2 was prepared from the purified DNA fragment and designated as pGEM-M2H.

### Example 2

### DNA Sequencing

The nucleotide sequence of the cDNA encoding region in the aforementioned plasmids was determined using Auto DNA Sequencer (Applied Biosystem) and ABI PRISM Dye Terminator Cycle Sequencing Ready Reaction Kit (Applied Biosystem) according to the manufacturer's protocol.

The nucleotide sequence of the gene coding the L chain V region from the mouse MABL-1 antibody, which is included in the plasmid pGEM-M1L, is shown in SEQ ID No. 5.

The nucleotide sequence of the gene coding the H chain V region from the mouse MABL-1 antibody, which is included in the plasmid pGEM-M1H, is shown in SEQ ID No. 6.

The nucleotide sequence of the gene coding the L chain V region from the mouse MABL-2 antibody, which is included in the plasmid pGEM-M2L, is shown in SEQ ID No. 7.

The nucleotide sequence of the gene coding the H chain V region from the mouse MABL-2 antibody, which is included in the plasmid pGEM-M2H, is shown in SEQ ID No. 8.

### Example 3

### Determination of CDR

The V regions of L and H chains generally have a similarity in their structures and each four framework regions therein are linked by three hypervariable regions, i.e., complementatarity determining regions (CDR). An amino acid sequence of the framework is relatively well conserved, while an amino acid sequence of CDR has extremely high variation (Kabat, E. A., et al., “Sequences of Proteins of Immunological Interest”, US Dept. Health and Human Services, 1983).

On the basis of these facts, the amino acid sequences of the variable regions from the mouse monoclonal antibodies to human IAP were applied to the database of amino acid sequences of the antibodies made by Kabat et al. and the homology thereof was investigated to determine the CDR. The results are shown in Table 1.

TABLE 1

| Plasmid | SEQ ID No. | CDR(1) | CDR(2) | CDR(3) |
|---|---|---|---|---|
| pGEM-M1L | 5 | 43-58 | 74-80 | 113-121 |
| pGEM-M1H | 6 | 50-54 | 69-85 | 118-125 |
| pGEM-M2L | 7 | 43-58 | 74-80 | 113-121 |
| pGEM-M1H | 8 | 50-54 | 69-85 | 118-125 |

### Example 4

### Identification of Cloned cDNA (Preparation of Chimera MABL-1 and MABL-2 Antibodies)

4.1 Preparation of a Vector Expressing Chimera MABL-1Antibody

cDNA clones, pGEM-M1L and pGEM-M1H, encoding the V regions of the L chain and the H chain of the mouse MABL-1 antibody, respectively, were modified by the PCR method and introduced into the HEF expression vector (WO92/19759) in order to prepare a vector expressing chimera MABL-1 antibody.

A forward primer MLS (SEQ ID No. 9) for the L chain V region and a forward primer MHS (SEQ ID No. 10) for the H chain V region were designed to hybridize to a DNA encoding the beginning of the leader sequence of each V region and to contain the Kozak consensus sequence (J. Mol. Biol., 196, 947-950, 1987) and HindIII restriction enzyme site. A reverse primer MLAS (SEQ ID No. 11) for the L chain V region and a reverse primer MHAS (SEQ ID No. 12) for the H chain V region were designed to hybridize to a DNA encoding the end of the J region and to contain the splice donor sequence and BamHI restriction enzyme site.

100 μl of a PCR solution comprising 10 Al of 10×PCR Buffer II, 2 mM MgCl₂, 0.16 mM dNTPs (DATP, dGTP, dCTP and dTTP), 5 units of DNA polymerase AmpliTaq Gold, 0.4 μM each of primers and 8 ng of the template DNA (pGEM-M1L or pGEM-M1H) was preheated at 94° C. of the initial temperature for 9 minutes and then heated at 94° C. for 1 minute, at 60° C. for 1 minute and at 72° C. for 1 minute in this order. This temperature cycle was repeated 35 times and then the reaction mixture was further heated at 72° C. for 10 minutes.

The PCR product was purified using the QIAquick PCR Purification Kit (QIAGEN) and then digested with HindIII and BamHI. The product from the L chain V region was cloned into the HEF expression vector, HEF-K and the product from the H chain V region was cloned into the HEF expression vector, HEF-γ. After DNA sequencing, plasmids containing a DNA fragment with a correct DNA sequence are designated as HEF-M1L and HEF-M1H, respectively.

4.2 Preparation of a Vector Expressing Chimera MABL-2 Antibody

Modification and cloning of CDNA were performed in the same manner described in Example 4.1 except for amplifying pGEM-M2L and pGEM-M2H as template DNA instead of pGEM-M1L and pGEM-M1H. After DNA sequencing, plasmids containing a DNA fragment with a correct DNA sequence are designated as HEF-M2L and HEF-M2H, respectively.

4.3 Transfection to COS7 Cell

The Expression of the aforementioned expression vectors were tested in COS7 cell to observe the transient expression of the chimera MABL-1 and MABL-2 antibodies.

(1) Transfection with a Gene Coding the Chimera MABL-1 Antibody

COS7 cells were co-transformed with the HEF-M1L and HEF-M1H vectors by an electroporation using the Gene Pulser apparatus (BioRad). Each DNA (10 μg) and 0.8 ml of 1×10⁷ cells/ml in PBS were added to a cuvette and pulse was given at 1.5 kV, 25 μF of electric capacity.

After the restoration for 10 minutes at room temperature, the electroporated cells were transferred into DMEM culture media (GIBCO BRL) containing 10% γ-globulin free fetal bovine serum. After culturing for 72 hours, the cultured supernatant was collected and cell shard was removed by centrifugation to obtain the recovered supernatant.

(2) Transfection with a Gene Coding the Chimera MABL-2 Antibody

The co-transfection to COS7 cells with the gene coding the chimera MABL-2 antibody was carried out in the same manner as described in Example 4.3-(1) except for using the HEF-M2L and HEF-M2H vectors instead of the HEF-M1L and HEF-M1H vectors to obtain the recovered supernatant.

4.4 Flow Cytometry

Flow cytometry was performed using the aforementioned COS7 cells cultured supernatant in order to measure binding to the antigen. The cultured supernatant of the COS7 cells expressing the chimera MABL-1 antibody or the COS7 cells expressing the chimera MABL-2 antibody, or human IgG antibody (SIGMA) as a control was added to 4×10⁵ cells of mouse leukemia cell line L1210 expressing human IAP and incubated on ice. After washing, the FITC-labeled anti-human IgG antibody (Cappel) was added thereto. After incubating and washing, the fluorescence intensity thereof was measured using the FACScan apparatus (BECTON DICKINSON).

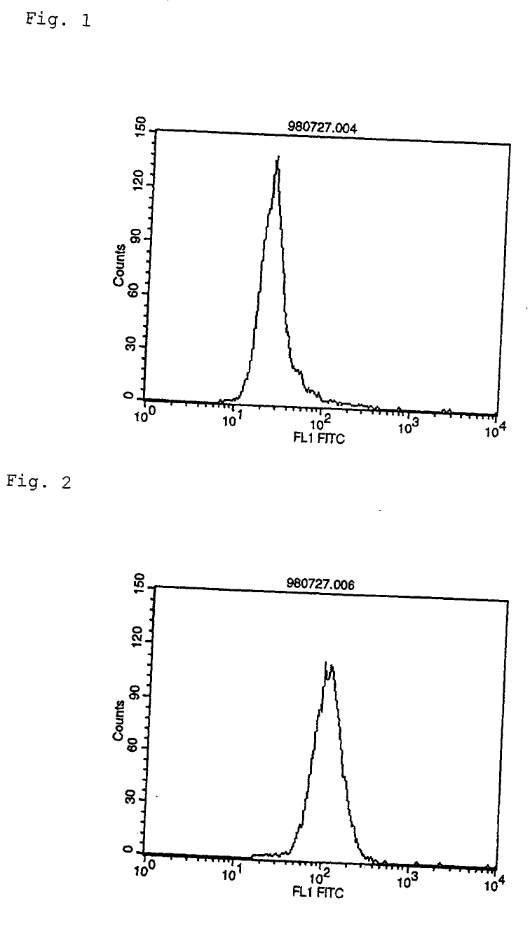

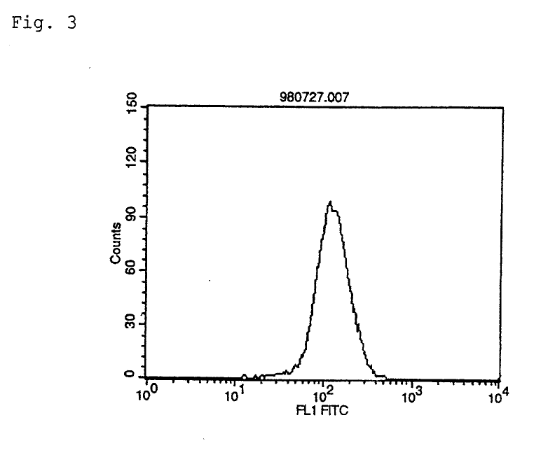

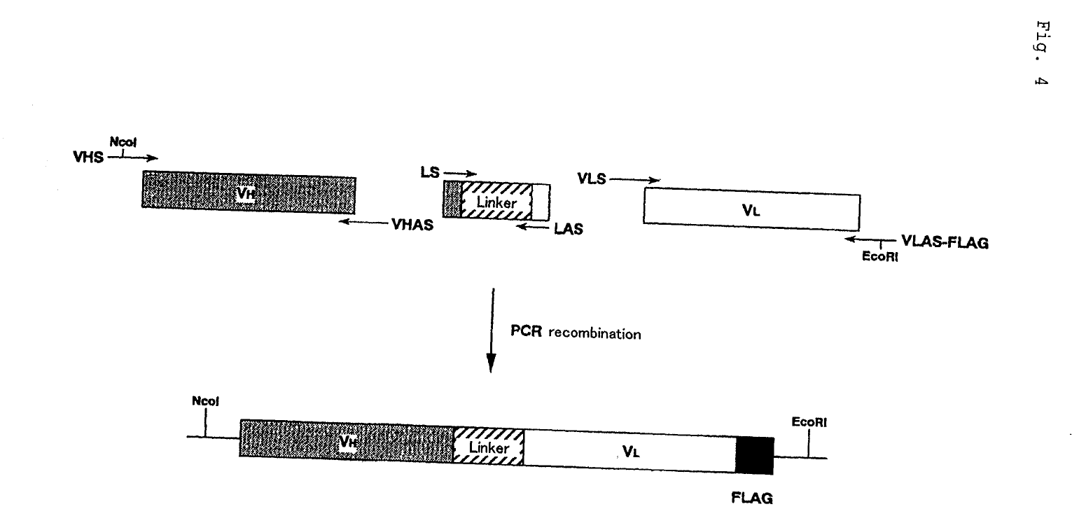

Since the chimera MABL-1 and MABL-2 antibodies were specifically bound to L1210 cells expressing human IAP, it is confirmed that these chimera antibodies have proper structures of the V regions of the mouse monoclonal MABL-1 and MABL-2 antibodies, respectively (FIGS. 1-3).

### Example 5

### Preparation of Single-Chain Fv (scFv) of the Reconstructed MABL-1 and MABL-2 Antibodies

5.1 Preparation of Reconstructed Single-Chain Fv of MABL-1 Antibody

The reconstructed single-chain Fv of MABL-1 antibody was prepared as follows. The H chain V region and the L chain V of MABL-1 antibody, and a linker were respectively amplified by the PCR method and were connected to produce the single-chain Fv of MABL-1 antibody. The production method is illustrated in FIG. 4. Six primers (A-E) were employed for the production of the single-chain Fv of MABL-1 antibody. Primers A, C and E have a sense sequence and primers B, D and F have an antisense sequence.

The forward primer VHS (Primer A, SEQ ID No. 13) for the H chain V region was designed to hybridize to a DNA encoding the N-terminal of the H chain V region and to contain Nco I restriction enzyme recognition site. The reverse primer VHAS (Primer B, SEQ ID No. 14) was designed to hybridize to a DNA coding the C-terminal of the H chain V region and to overlap with the linker.

The forward primer LS (Primer C, SEQ ID No. 15) for the linker was designed to hybridize to a DNA encoding the N-terminal of the linker and to overlap with a DNA encoding the C-terminal of the H chain V region. The reverse primer LAS (Primer D, SEQ ID No. 16) was designed to hybridize to a DNA encoding the C-terminal of the linker and to overlap with a DNA encoding the N-terminal of the L chain V region.

The forward primer VLS (Primer E, SEQ ID No. 17) for the L chain v region was designed to hybridize to a DNA encoding the C-terminal of the linker and to overlap with a DNA encoding the N-terminal of the L chain V region. The reverse primer VLAS-FLAG (Primer F, SEQ ID No. 18) was designed to hybridize to a DNA encoding the C-terminal of the L chain V region and to have a sequence coding the FLAG peptide, two stop codons and EcoRI restriction enzyme recognition site.

In the first PCR step, three reactions, A-B, C-D and E-F, were carried out and PCR products thereof were purified. Three PCR products obtained from the first PCR step were assembled by their complementarity. Then, the primers A and F were added to them and a full length DNA encoding the single-chain Fv of MABL-1 antibody was amplified (Second PCR). In the first PCR, a plasmid pGEM-M1H coding the H chain V region of MABL-1 antibody (see Example 2), a plasmid pSC-DP1 which comprises a DNA sequence coding a linker region comprising: Gly Gly Gly Gly Ser Gly Gly Gly Gly Ser Gly Gly Gly Gly Ser (SEQ ID No. 19) (Huston, J. S., et al., Proc. Natl. Acad. Sci. USA, 85, 5879-5883, 1988) and the plasmid pGEM-MlL coding the L chain V region of MABL-1 antibody (see Example 2) were employed as a template, respectively.

50 μl of the solution for the first PCR step comprises 5 μl of 10×PCR Buffer II, 2 mM MgCl₂, 0.16 mM dNTPs, 2.5 units of DNA polymerase, AmpliTaq Gold (PERKIN ELMER, respectively), 0.4 μM of each primers and 5 ng of each template DNA. The PCR solution was preheated at 94° C. of the initial temperature for 9 minutes and then heated at 94° C. for 1 minute, at 65° C. for 1 minute and at 72° C. for 1 minute and 20 seconds in this order. This temperature cycle was repeated 35 times and then the reaction mixture was further heated at 72° C. for 7 minutes.

The PCR products A-B (371 bp), C-D (63 bp) and E-F (384 bp) were purified using the QIAquick PCR Purification Kit (QIAGEN) and were assembled for the second PCR. In the second PCR, 98 μl of a PCR mixture comprising 120 ng of the first PCR product A-B, 20 ng of the PCR product C-D and 120 ng of the PCR product E-F, 10 μl of 10×PCR Buffer II, 2 mM MgCl₂, 0.16 mM dNTPs, 5 units of DNA polymerase AmpliTaq Gold (PERKIN ELMER) was preheated at 94° C. of the initial temperature for 8 minutes and then heated at 94° C. for 2 minutes, at 65° C. for 2 minutes and at 72° C. for 2 minutes in this order. This temperature cycle was repeated twice and then 0.4 μM each of primers A and F were added into the reaction, respectively. The mixture was preheated at 94° C. of the initial temperature for 9 minutes and then heated at 94° C. for 1 minute, at 60° C. for 1 minute and at 72° C. for 1 minute and 20 seconds in this order. This temperature cycle was repeated 35 times and then the reaction mixture was further heated at 72° C. for 7 minutes.

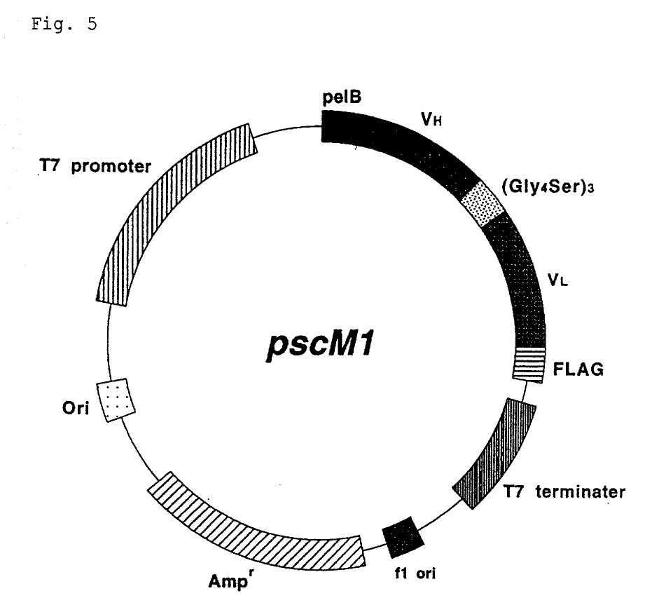

A DNA fragment of 843 bp produced by the second PCR was purified and digested by NcoI and EcoRI. The resultant DNA fragment was cloned into pSCFVT7 vector. The expression vector pSCFVT7 contains a pelB signal sequence suitable for E. coli periplasmic expression system (Lei, S. P., et al., J. Bacteriology, 169, 4379-4383, 1987). After the DNA sequencing, a plasmid containing a DNA fragment encoding a correct amino acid sequence of the single-chain Fv of MABL-1 antibody is designated as “pscM1” (see FIG. 5). A nucleotide sequence and an amino acid sequence of the single-chain Fv of MABL-1 antibody contained in the plasmid pscM1 are shown in SEQ ID No. 20.

pscM1 vector was modified by the PCR method in order to prepare a vector expressing the single-chain Fv of MABL-1 antibody in mammal cells. The resultant DNA fragment was introduced into pCHO1 expression vector. This expression vector, pCHO1, is constructed in the manner that an antibody gene is excluded from DHFR-ΔE-rvH-PM1-f (WO92/19759) by digesting with EcoRI and SmaI and the EcoRI-NotI-BamHI Adapter (Takara shuzo) is linked thereto.

As a forward primer for PCR, Sal-VHS primer shown in SEQ ID No. 21 was designed to hybridize to a DNA encoding the N-terminal of the H chain V region and to contain SalI restriction enzyme recognition site. As a reverse primer for PCR, FRH1-anti primer shown in SEQ ID No. 22 was designed to hybridize to a DNA encoding the end of the first framework sequence.

100 μl of the solution comprising 10 μl of 10×PCR Buffer II, 2 mM MgCl₂, 0.16 mM dNTPs, 5 units of the DNA polymerase, AmpliTaq Gold, 0.4 μl M of each primer and 8 ng of a template DNA (pscM1) was preheated at 95° C. of the initial temperature for 9 minutes and then heated at 95° C. for 1 minute, at 60° C. for 1 minute and at 72° C. for 1 minute and 20 seconds in this order. This temperature cycle was repeated 35 times and then the reaction mixture was further heated at 72° C. for 7 minutes.

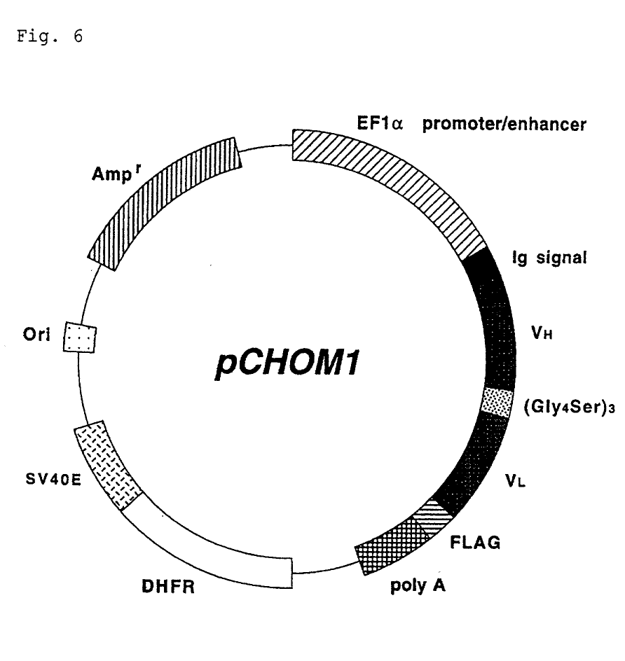

The PCR product was purified using the QIAquick PCR Purification Kit (QIAGEN) and digested by SalI and MboII to obtain a DNA fragment encoding the N-terminal of the single-chain Fv of MABL-1 antibody. The pscMl vector was digested by MboII and EcoRI to obtain-a DNA fragment encoding the C-terminal of the single-chain Fv of MABL-1 antibody. The SalI-MboII DNA fragment and the MboII-EcoRI DNA fragment were cloned into pCHO1-Igs vector. After DNA sequencing, a plasmid comprising the desired DNA sequence is designated as “pCHOM1” (see FIG. 6). The expression vector, pCHO1-Igs, contains a mouse IgG1 signal sequence suitable for the secretion-expression system in mammal cells (Nature, 322, 323-327, 1988). A nucleotide sequence and an amino acid sequence of the single-chain Fv of MABL-1 antibody contained in the plasmid pCHOM1 are shown in SEQ ID No. 23.

5.2 Preparation of Reconstructed Single-Chain Fv of MABL-2 Antibody

The reconstructed single-chain Fv of MABL-2 antibody was prepared in accordance with the aforementioned Example 5.1. Employed in the first PCR step were a plasmid pGEM-M2H coding the H chain V region of MABL-2 (see Example 2) instead of pGEM-M1H and a plasmid pGEM-M2L coding the L chain V region of MABL-2 (see Example 2) instead of pGEM-M1L, to obtain a plasmid pscM2 which comprises a DNA fragment encoding the desired amino acid sequence of the single-chain Fv of MABL-2 antibody. A nucleotide sequence and an amino acid sequence of the single-chain Fv of MABL-2 antibody contained in the plasmid pscM2 are shown in SEQ ID No. 24.

pscM2 vector was modified by the PCR method to prepare a vector, pCHOM2, for the expression in mammal cells which contains a DNA fragment encoding the desired amino acid sequence of the single-chain Fv of MABL-2 antibody. A nucleotide sequence and an amino acid sequence of the single-chain Fv of MABL-2 antibody contained in the pCHOM2 plasmid are shown in SEQ ID No. 25.

5.3 Transfection to COS7 Cells

The pCHOM2 vector was tested in COS7 cells in order to observe the transient expression of the reconstructed single-chain Fv of MABL-2 antibody.

The COS7 cells were transformed with the pCHOM2 vector by electroporation using the Gene Pulser apparatus (BioRad). The DNA (10 μg) and 0.8 ml of 1×10⁷ cells/ml in PBS were added to a cuvette and pulse was given at 1.5 kV, 25 μF of electric capacity.

After the restoration for 10 minutes at room temperature, the electroporated cells were transferred into the IMDM culture media (GIBCO BRL) containing 10% fetal bovine serum. After culturing for 72 hours, the cultured supernatant was collected and cell shard was removed by centrifugation to obtain the withdrawn supernatant.

5.4 Detection of the Reconstructed Single-Chain Fv of MABL-2 Antibody in Cultured Supernatant of COS7 Cell

The existence of the single-chain Fv of MABL-2 antibody in the cultured supernatant of COS7 cells which had been transfected with the pCHOM2 vector was confirmed by the Western Blotting method.

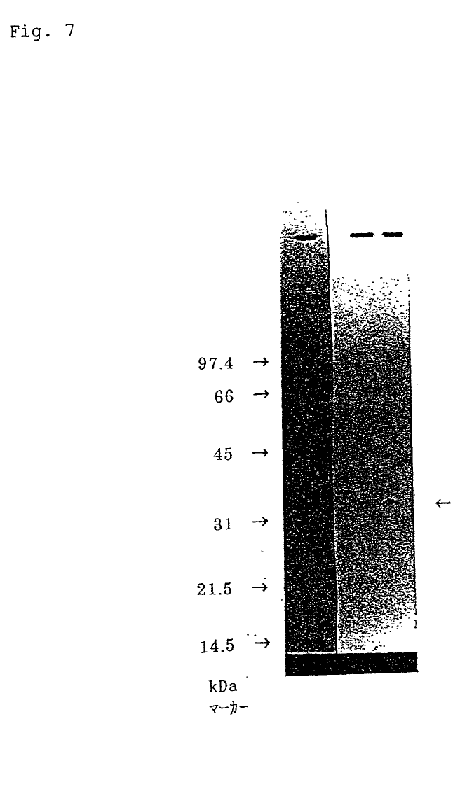

The cultured supernatant of COS7 cells transfected with the pCHOM2 vector and the cultured supernatant of COS7 cells transfected with the pCHO1 as a control were subjected to SDS electrophoresis and transferred to REINFORCED NC membrane (Schleicher & Schuell). The membrane was blocked with 5% skim milk (Morinaga Nyu-gyo) and washed with 0.05% Tween 20-PBS. Subsequently, an anti-FLAG antibody (SIGMA) was added thereto. The membrane was incubated at room temperature and washed and then the alkaline phosphatase-conjugated mouse IgG antibody (Zymed) was added. After the incubation and washing at room temperature, the substrate solution (Kirkegaard Perry Laboratories) was added and chromogenized (FIG. 7).

The FLAG-peptide specific protein was detected only in the cultured supernatant of the pCHO1 vector-introduced COS7 cells and thus it is confirmed that the single-chain Fv of MABL-2 antibody was secreted in this cultured supernatant.

5.5 Flow Cytometry

Flow cytometry was performed using the aforementioned COS7 cells cultured supernatants in order to measure the binding to the antigen. The cultured supernatant of the COS7 cells expressing the single-chain Fv of MABL-2 antibody or the cultured supernatant of COS7 cells transformed with pCHO1 as a control was added to 2×10⁵ cells of the mouse leukemia cell line L1210 expressing human Integrin Associated Protein (IAP) or the cell line L1210 transformed with pCOS1 as a control. After incubation on ice and washing, the mouse anti-FLAG antibody (SIGMA) was added and then the cells were incubated and washed. Then, the FITC labeled anti-mouse IgG antibody (BECTON DICKINSON) was added thereto and the cells were incubated and washed again. Subsequently, the fluorescence intensity was measured using the FACScan apparatus (BECTON DICKINSON).

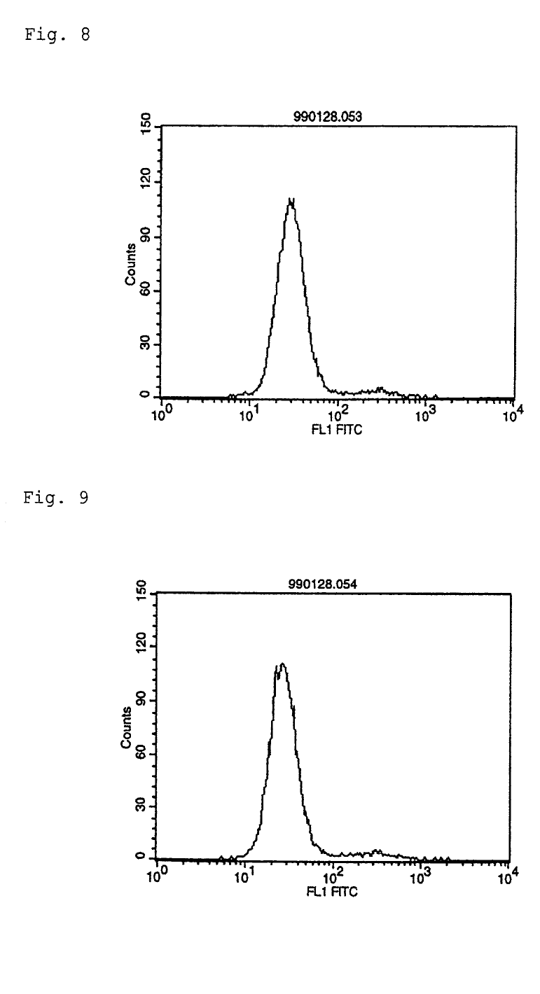

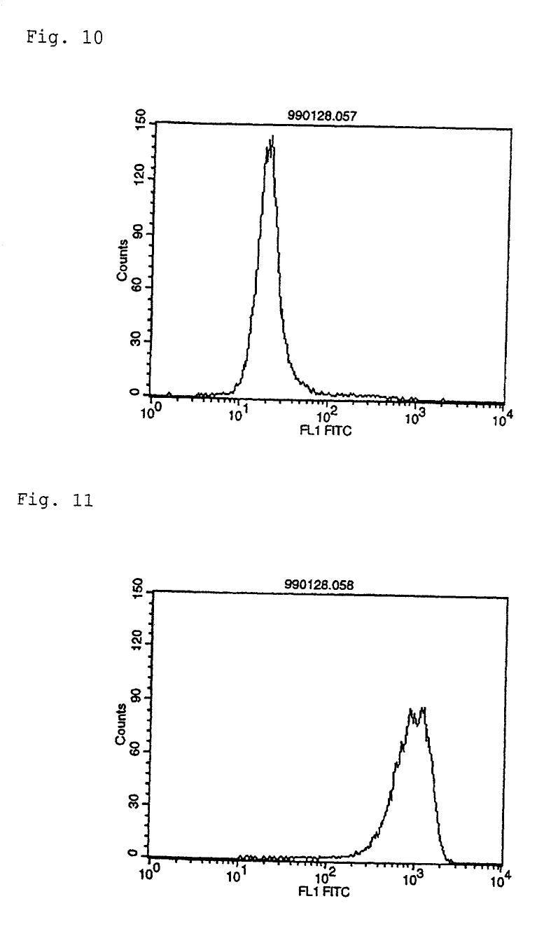

Since the single-chain Fv of MABL-2 antibody was specifically bound to L1210 cells expressing human IAP, it is confirmed that the single-chain Fv of MABL-2 antibody has an affinity to human Integrin Associated Protein (IAP) (see FIGS. 8-11).

5.6 Competitive ELISA

The binding activity of the single-chain Fv of MABL-2 antibody was measured using the inhibiting activity against the binding of mouse monoclonal antibodies to the antigen as an index.

The anti-FLAG antibody adjusted to 1 μg/ml was added to each well on 96-well plate and incubated at 37° C. for 2 hours. After washing, blocking was performed with 1% BSA-PBS. After the incubation at room temperature and washing, the cultured supernatant of COS7 cells into which the soluble human IAP antigen gene (SEQ ID No. 26) had been introduced was diluted twice with PBS and added to each well. After incubation at room temperature and washing, a mixture of 50 μl of the biotinized MABL-2 antibody adjusted to 100 ng/ml and 50 μl of sequentially diluted supernatant of the COS7 cells expressing the single-chain Fv of MABL-2 antibody was added into each well, incubated at room temperature and washed. Then, the alkaline phosphatase-conjugated streptoavidin (Zymed) was added into each well. After incubation at room temperature and washing, the substrate solution (SIGMA) was added and an absorbance at 405 nm of the reaction mixture in each well was measured.

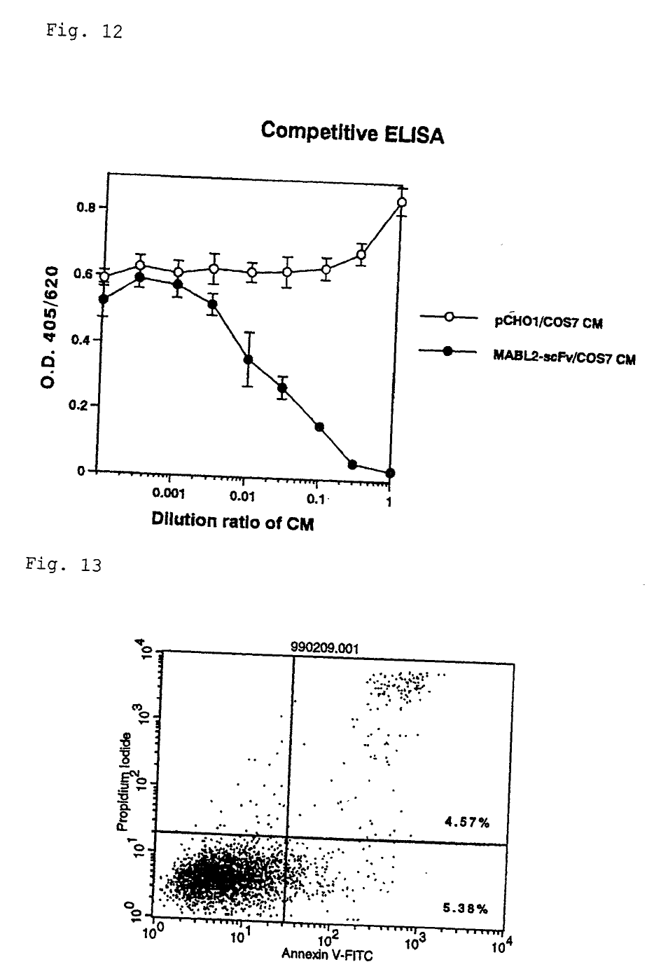

The results revealed that the single-chain Fv of MABL-2 antibody (MABL2-scFv) remarkably inhibited the binding of the mouse MABL-2 antibody to human IAP antigen dependent on the concentration thereof in comparison with the cultured supernatant of the PCHO1-introduced COS7 cells as a control (FIG. 12). Accordingly, it is suggested that the single-chain Fv of MABL-2 antibody has a correct construction of each of the V regions from the mouse monoclonal antibody MABL-2.

5.7 Apoptosis-Inducing Effect in vitro

An apoptosis-inducing action of the single-chain Fv of MABL-2 antibody was examined by Annexin-V staining (Boehringer Mannheim) using the L1210 cells transfected with human IAP gene, the L1210 cells transfected with the pCOS1 vector as a control and CCRF-CEM cells.

To each 1×10⁵ cells of the above cells was added a cultured supernatant of the COS7 cells expressing the single-chain Fv of MABL-2 antibody or a cultured supernatant of COS7 cells transfected with the pCHO1 vector as a control at 50% final concentration and the mixtures were cultured for 24 hours. Then, the Annexin-V staining was performed and the fluorescence intensity was measured using the FACScan apparatus (BECTON DICKINSON).

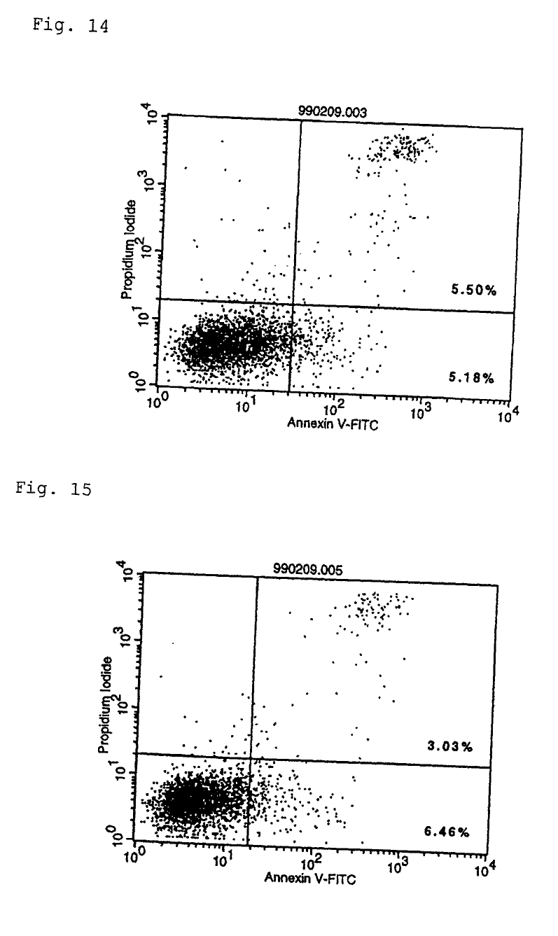

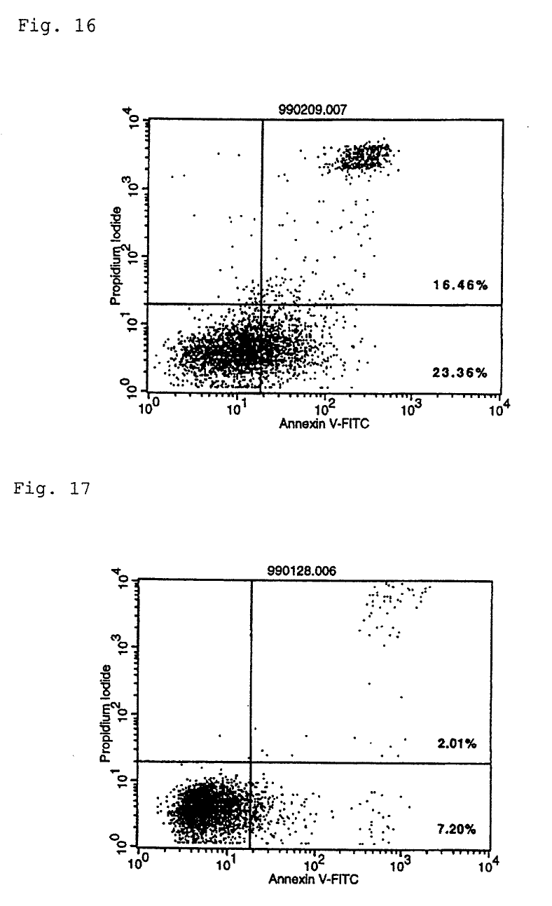

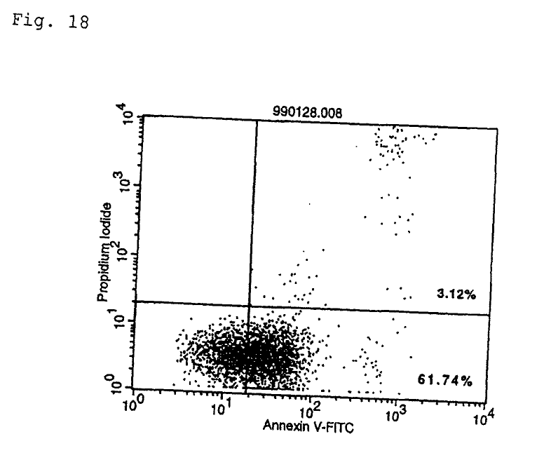

Results of the Annexin-V staining are shown in FIGS. 13-18, respectively. Dots in the left-lower region represent living cells and dots in the right-lower regions represent cells at the early stage of apoptosis and dots in the right-upper region represents cells at the late stage of apoptosis. The results show that the single-chain Fv of MABL-2 antibody (MABL2-scFv) remarkably induced human IAP specific cell death of L1210 cells (FIGS. 13-16) and that the single-chain Fv also induced cell death of CCRF-CEM cells in comparison with the control (FIGS. 17-18).

5.8 Expression of MABL-2 Derived Single-Chain Fv in CHO Cells

CHO cells were transfected with the pCHOM2 vector in order to establish a CHO cell line which is continuously expressing the single-chain Fv derived from the MABL-2 antibody.

CHO cells were transformed with the pCHOM2 vector by the electroporation using the Gene Pulser apparatus (BioRad). A mixture of DNA (10 μg) and 0.7 ml of CHO cell (1×10⁷ cells/ml) suspended in PBS was added to a cuvette and pulse was given at 1.5 kV, 25 μF of electric capacity. After the restoration for 10 minutes at room temperature, the electroporated cells were transferred into nucleic acid free α-MEM media (GIBCO BRL) containing 10% fetal bovine serum and cultured. The expression of desired protein in the resultant clones was confirmed by SDS-PAGE and a clone with a high expression level was selected as a cell line producing the single-chain Fv derived from the MABL-2 antibody. The cell line was cultured in serum free medium CHO-S-SFM II (GIBCO BRL) containing 10 nM methotrexate (SIGMA). Then, the cultured supernatant was collected and cell residue was removed by centrifugation to obtain the withdrawn supernatant.

5.9 Purification of MABL-2 Derived Single-Chain Fv Produced in CHO Cells

The cultured supernatant of the CHO cell line expressing the single-chain Fv (polypeptide) obtained in Example 5.8 was concentrated up to twenty times using a cartridge for the artificial dialysis (PAN130SF, ASAHI MEDICALS). The concentrated solution was stored at −20° C. and thawed on purification.

Purification of the single-chain Fv from the cultured supernatant of the CHO cells was performed using three kinds of chromatography, i.e., Blue-sepharose chromatography, a hydroxyapatite chromatography and a gel filtration chromatography.

(1) Blue-Sepharose Column Chromatography

The concentrated solution of the supernatant was diluted to ten times with 20 mM acetate buffer (pH 6.0) and insoluble materials were removed by centrifugation (10000 rpm×30 minutes). The supernatant was applied onto a Blue-sepharose column (20 ml) equilibrated with the same buffer. After washing the column with the same buffer, proteins adsorbed to the column were eluted by a stepwise gradient of NaCl in the buffer, 0.1, 0.2, 0.3, 0.5 and up to 1.0 M. The unbound fraction and each eluted fraction were analyzed by SDS-PAGE. The fractions in which the single-chain Fv was confirmed (the fractions eluted at 0.1 to 0.3M NaCl) were pooled and concentrated up to approximately 20 times using CentriPrep-10 (AMICON).

(2) Hydroxyapatite Column Chromatography

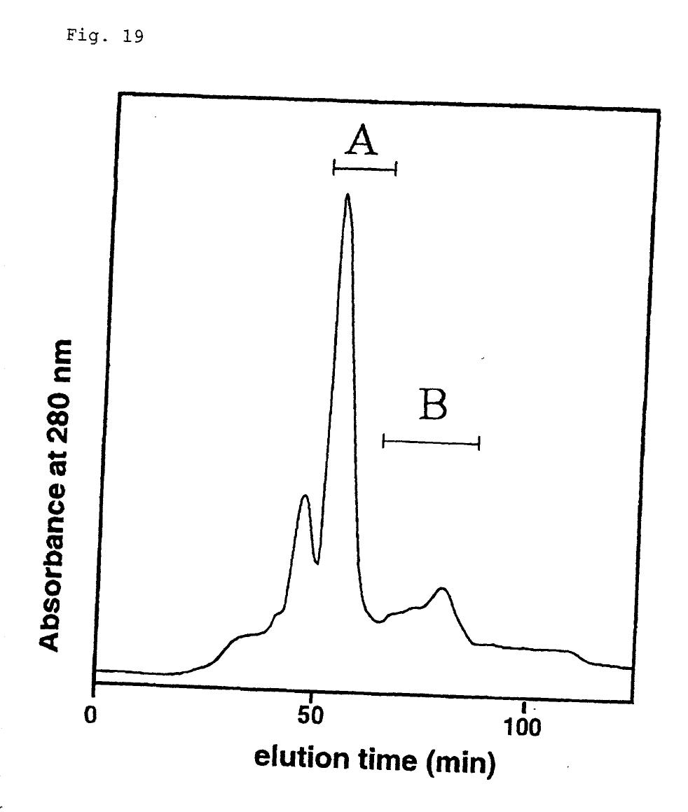

The concentrated solution obtained in (1) was diluted to 10 times with 10 mM phosphate buffer (pH 7.0) and applied onto the hydroxyapatite column (20 ml, BIORAD). The column was washed with 60 ml of 10 mM phosphate buffer (pH 7.0). Then, proteins adsorbed to the column were eluted by a linear gradient of sodium phosphate from 10 to 200 mM (see FIG. 19). The analysis of each fraction by SDS-PAGE confirmed the single-chain Fv in fractions A and B.

(3) Gel Filtration

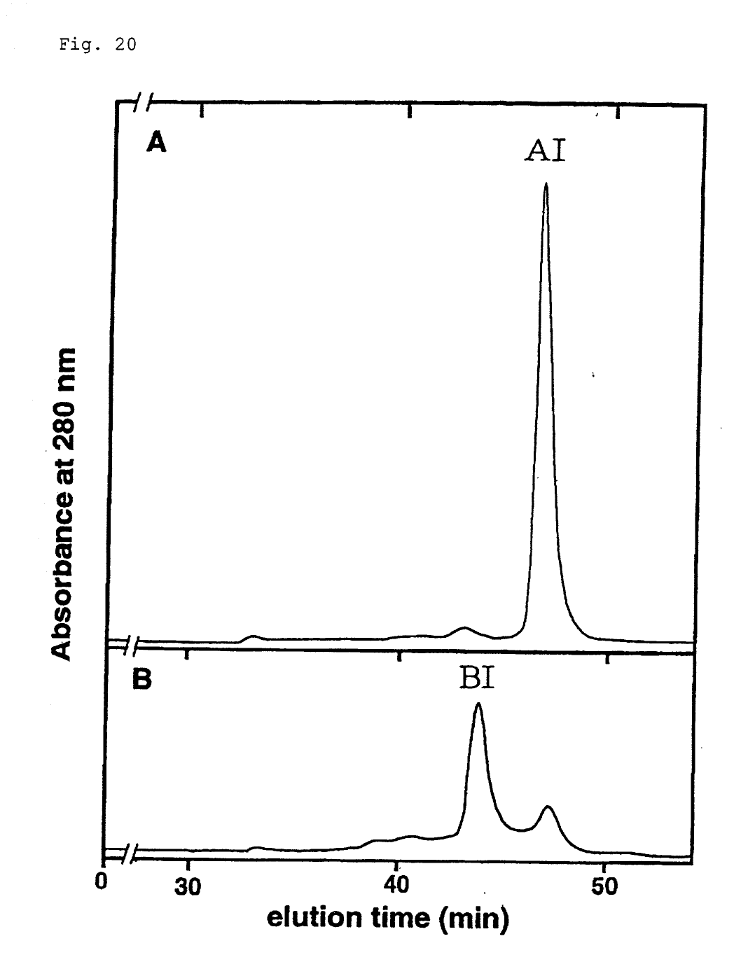

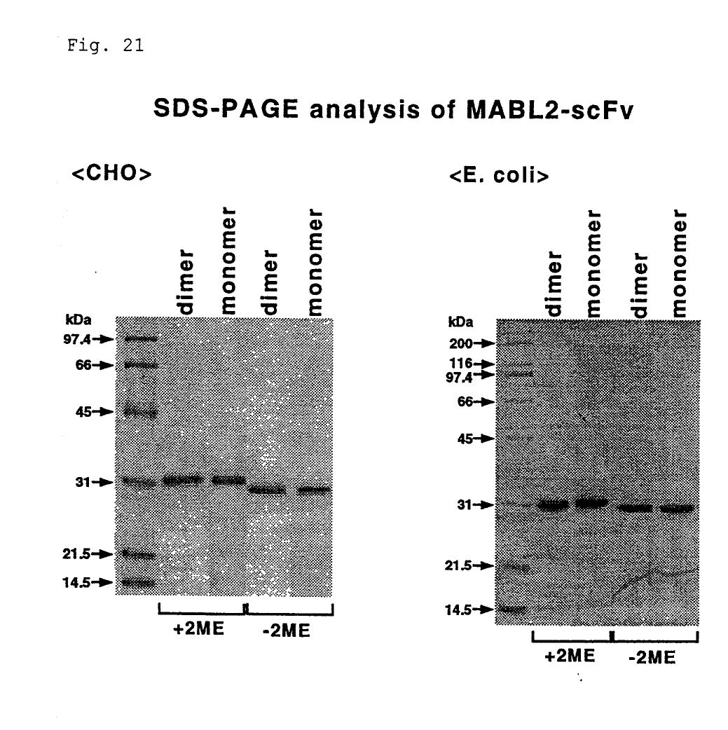

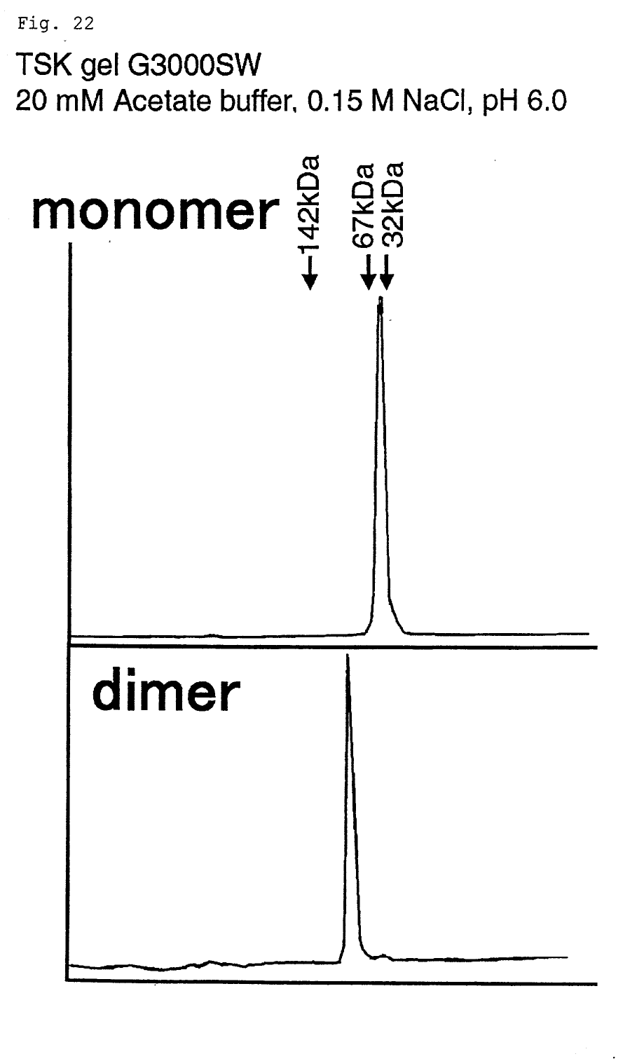

Each of fractions A and B in (2) was separately concentrated with CentriPrep-10 and applied onto TSKgel G3000SWG column (21.5×600 mm) equilibrated with 20 mM acetate buffer (pH 6.0) containing 0.15 M NaCl. Chromatograms are shown in FIG. 20. The analysis of the fractions by SDS-PAGE confirmed that both major peaks (AI and BI) are of desired single-chain Fv. In the gel filtration analysis, the fraction A was eluted at 36 kDa of apparent molecular weight and the fraction B was eluted at 76 kDa. The purified single-chain Fvs (AI, BI) were analyzed with 15% SDS polyacrylamide gel. Samples were treated in the absence or presence of a reductant and the electrophoresis was carried out in accordance with the Laemmli's method. Then, the protein was stained with Coomassie Brilliant Blue. As shown in FIG. 21, both AI and BI give a single band at 35 kDa of apparent molecular weight, regardless of the absence or presence of the reductant. From the above results, it is expected that AI is a monomer of the single-chain Fv and BI is a non-covalent dimer of the single-chain Fv. The gel filtration analysis of the fractions AI and BI with TSKgel G3000SW column (7.5×60 mm) reveals that a peak of the monomer is detected only in the fraction AI and a peak of the dimer is detected only in the fraction BI (FIG. 22).

5.10 Construction of Vector Expressing Single-Chain Fv Derived from MABL-2 Antibody in E. coli Cell

pscM2 vector was modified by the PCR method in order to prepare a vector effectively expressing the single-chain Fv from the MABL-2 antibody in E. coli cells. The resultant DNA fragment was introduced into pSCFVT7 expression vector.

As a forward primer for PCR, Nde-VHSm02 primer shown in SEQ ID No. 27 was designed to hybridize to a DNA encoding the N-terminal of the H chain V region and to contain a start codon and NdeI restriction enzyme recognition site. As a reverse primer for PCR, VLAS primer shown in SEQ ID No. 28 was designed to hybridize to a DNA encoding the C-terminal of the H chain V region and to contain two stop codons and EcoRI restriction enzyme recognition site. The forward primer, Nde-VHSm02, comprises five point mutations in the part hybridizing to the DNA encoding the N-terminal of the H chain V region for the effective expression in E. coli.

100 μl of a PCR solution comprising 10 μl of 10×PCR Buffer #1, 1 mM MgCl₂, 0.2 mM dNTPs, 5 units of KOD DNA polymerase (all from TOYOBO), 1 μM of each primer and 100 ng of a template DNA (pscM2). The PCR solution was heated at 98° C. for 15 seconds, at 65° C. for 2 seconds and at 72° C. for 30 seconds in this order and this temperature cycle was repeated 25 times.

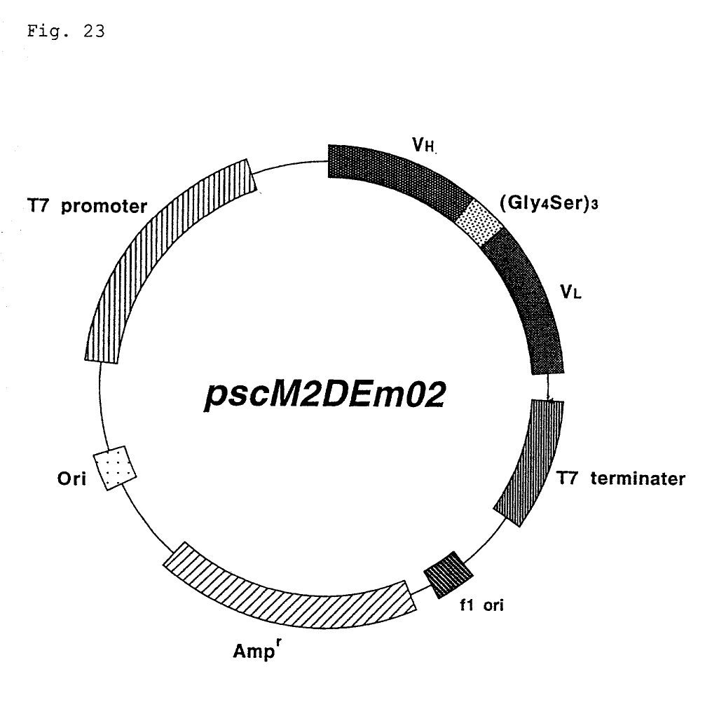

The PCR product was purified using the QIAquick PCR Purification Kit (QIAGEN) and digested by NdeI and EcoRI, and then the resulting DNA fragment was cloned into pSCFVT7 vector, from which pelB signal sequence had been excluded by the digestion with NdeI and EcoRI. After the DNA sequencing, the resulting plasmid comprising a DNA fragment with a desired DNA sequence is designated as “pscM2DEm02” (see FIG. 23). A nucleotide sequence and an amino acid sequence of the single-chain Fv derived from the MABL-2 antibody contained in the plasmid pscM2DEm02 are shown in SEQ ID No. 29.

5.11 Expression of Single-Chain Fv Derived from MABL-2 Antibody in E. coli Cells

E. coli BL21(DE3)pLysS (STRATAGENE) was transformed with pscM2DEm02 vector in order to obtain a strain of E. coli expressing the single-chain Fv derived from MABL-2 antibody. The resulting clones were examined for the expression of the desired protein using SDS-PAGE, and a clone with a high expression level was selected as a strain producing the single-chain Fv derived from MABL-2 antibody.

5.12 Purification of Single-Chain Fv Derived from MABL-2 Antibody Produced in E. coli

A single colony of E. coli obtained by the transformation was cultured in 3 ml of LB medium at 28° C. for 7 hours and the bacteria were transplanted to 70 ml of LB medium and cultured at 28° C. overnight. This pre-cultured medium was transplanted to 7 L of LB medium and cultured at 28° C. with stirring at 300 rpm using the Jar-fermenter. When an absorbance of the medium reached O.D.=1.5, the bacteria were induced with 1 mM IPTG and then cultured for 3 hours.

The cultured medium was centrifuged (10000 g×10 minutes) and the bacteria were withdrawn as precipitate. To the bacteria was added 50 mM Tris-HCl buffer (pH 8.0) containing 5 mM EDTA, 0.1 M NaCl and 1% Triton X-100 and the bacteria was disrupted by the ultrasonication (out put: 4, duty cycle: 70%, 1 minute×10 times). This disrupted suspension was centrifuged (12000 g×10 minutes) and an inclusion body was withdrawn as precipitate. To the inclusion body was added 50 mM Tris-HCl buffer (pH 8.0) containing 5 mM EDTA, 0.1 M NaCl and 4% Triton X-100 and the inclusion body was treated with the ultrasonication (out put: 4, duty cycle: 50%, 30 seconds×2 times) again and centrifuged (12000 g×10 minutes). Subsequently, the desired protein was withdrawn as precipitate and contaminated proteins included in the supernatant were removed.

The inclusion body comprising the desired protein was lysed in 50 mM Tris-HCl buffer (pH 8.0) containing 6 M Urea, 5 mM EDTA and 0.1 M NaCl and the lysate was applied onto Sephacryl S-300 gel filtration column (5×90 cm, Amersharm Pharmacia) equilibrated with 50 mM Tris-HCl buffer (pH 8.0) containing 4M Urea, 5 mM EDTA, 0.1 M NaCl and 10 mM mercaptoethanol at a flow rate of 5 ml/minutes to remove associated single chain Fvs with a high-molecular weight. The obtained fractions were studied with SDS-PAGE and the fractions with high purity of the protein were diluted with the buffer used in the gel filtration up to O.D₂₈₀=0.25. Then, the fractions were dialyzed three times against 50 mM Tris-HCl buffer (pH 8.0) containing 5 mM EDTA, 0.1 M NaCl, 0.5 M Arg, 2 mM glutathione in the reduced form and 0.2 mM glutathione in the oxidized form in order for the protein to be unwound. Further, the fraction was dialyzed three times against 20 mM acetate buffer (pH 6.0) containing 0.15 M NaCl, the buffer being exchanged at each time.

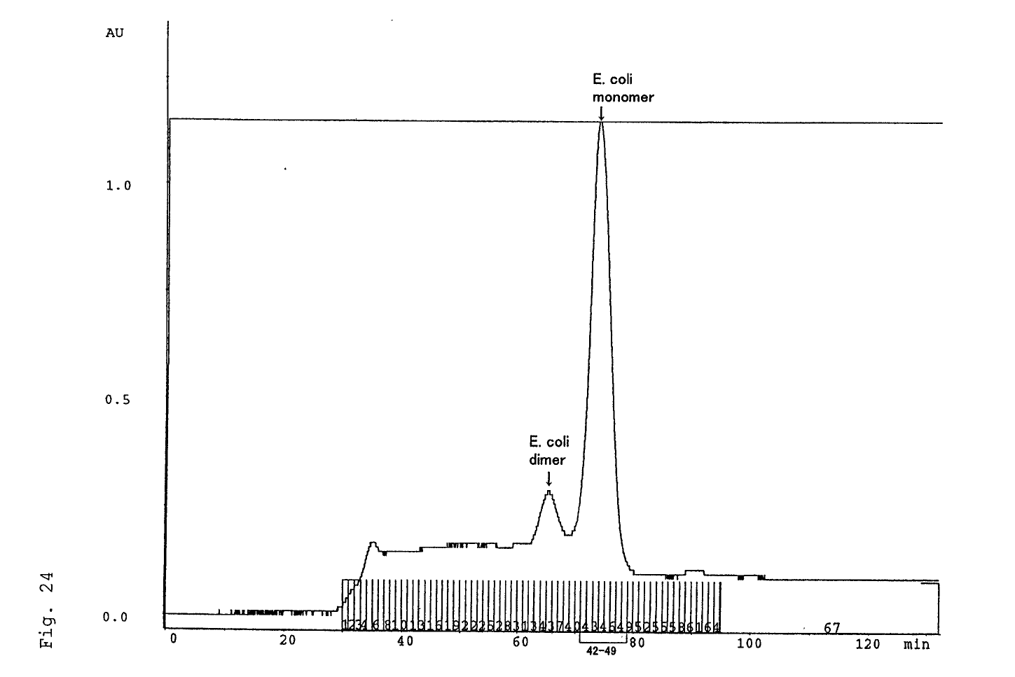

The dialysate was applied onto Superdex 200 pg gel filtration column (2.6×60 cm, Amersharm Pharmacia) equilibrated with 20 mM acetate buffer (pH 6.0) containing 0.15 M NaCl in order to remove high molecular weight proteins which were intermolecularly crosslinked by S—S bonds. As shown in FIG. 24, two peaks, major and sub peaks, were eluted after broad peaks, which are expected to be an aggregate of the high molecular weight protein. The analysis by SDS-PAGE (see FIG. 21) and the elution positions of the two peaks in the gel filtration analysis suggest that the major peak is of the monomer of the single-chain Fv and the sub peak is of the noncovalent dimer of the single-chain Fv.

5.13 Apoptosis Inducing Activity of Single-Chain Fv Derived from MABL-2 Antibody in vitro

An apoptosis inducing action of the single-chain Fv from MABL-2 antibody (MABL2-scFv) produced by the CHO cells or E. coli was examined by Annexin-V staining (Boehringer Mannheim) in the L1210 cells into which human IAP gene (hIAP-L1210) had been introduced.

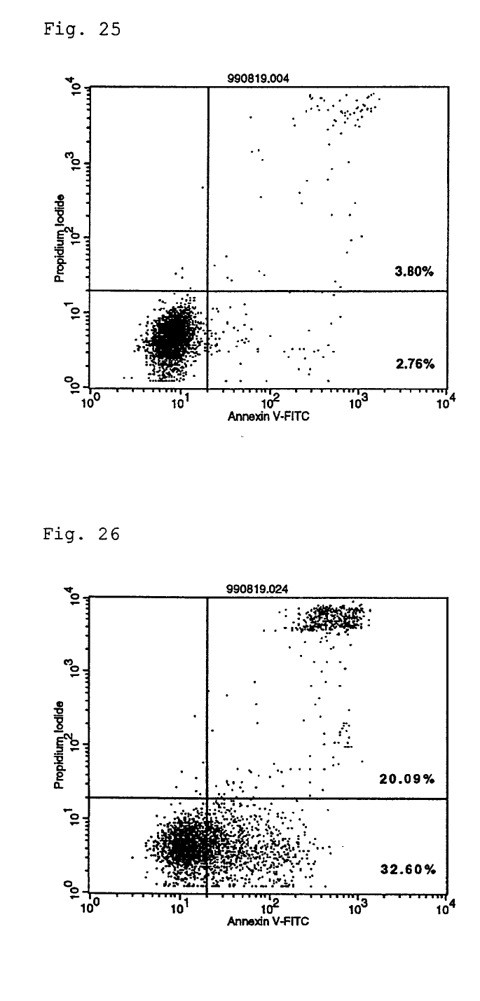

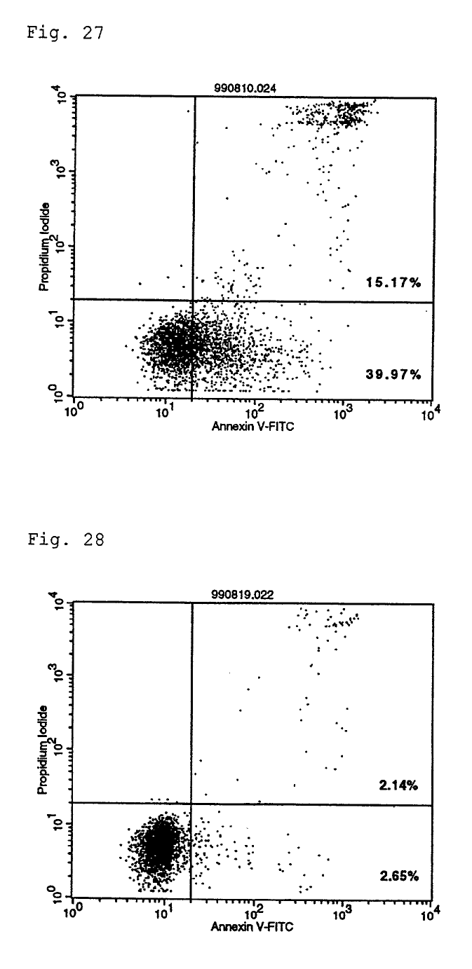

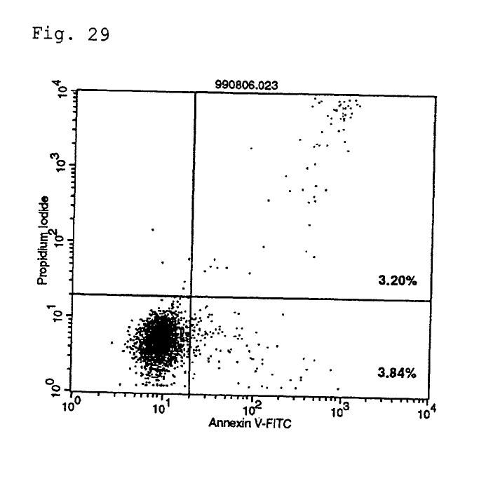

A sample antibody at the final concentration of 3 μg/ml was added to 5×10⁴ cells of hIAP/L1210 cell line and cultured for 24 hours. Sample antibodies, i.e., the monomer and the dimer of the single-chain Fv of MABL-2 from the CHO cells obtained in Example 5.9, the monomer and the dimer of the single-chain Fv of MABL-2 from E. coli obtained in Example 5.12, and the mouse IgG antibody as a control were analyzed. After culturing, the Annexin-V staining was carried out and the fluorescence intensity thereof was measured using the FACScan apparatus (BECTON DICKINSON). Results of the analysis by the Annexin-V staining are shown in FIGS. 25-29. The results shows that the dimers of the single-chain Fv polypeptide of MABL-2 produced in the CHO cells and E. coli (FIGS. 26, 27) remarkably induced cell death in comparison with the antibody of the control (FIG. 25), while no apoptosis inducing action was observed in the monomers of the single-chain Fv polypeptide of MABL-2 produced in the CHO cells and E. coli (FIGS. 28, 29).

5.14 Antitumor Effect of the Monomer and the Dimer of scFv/CHO Polypeptide for a Model Mouse of Human Myeloma

(1) Measurrement of Human IgG in Mouse Serum

Measurement of human IgG contained in mice was carried out-by the following ELISA. 100 μL of goat anti-human IgG antibody (BIOSOURCE, Lot#7902) diluted to 1 μg/mL with 0.1% bicarbonate buffer (pH 9.6) was added to each well on 96 wells plate and incubated at 4° C. overnight so that the antibody was made to form a solid phase. After blocking, 100 μL of the stepwise diluted mouse serum or the human IgG (CAPPEL, Lot#00915) as a standard was added to each well and incubated for 2 hours at room temperature. After washing, 100 μL of alkaline phosphatase-labeled anti-human IgG antibody (BIOSOURCE, Lot#6202) which had been diluted to 5000 times was added to the reaction mixtures, and incubation was carried out for 1 hour at room temperature. After washing, a substrate solution was added to the mixtures. After incubation, absorbance value at 405 nm of the reaction mixture in each well was measured using the MICROPLATE READER Model 3550 (BioRad) and the concentration of human IgG in the mouse serum was calculated in accordance with the measured calibration curve obtained from the absorbance values of human IgG as the standard.

(2) Preparation of Antibodies for Administration

The dimer and the monomer of the scFv/CHO polypeptide were respectively diluted to 0.4 mg/mL or 0.25 mg/mL with filtered PBS(−) at the day of administration to prepare samples for the administration.

(3) Preparation of a Model Mouse of Human Myeloma

A model mouse of human myeloma was prepared as follows. KPMM2 cell line passaged in vivo (JP-A-7-236475) with SCID mouse (Japan Clare) was suspended in RPMI1640 media (GIBCO-BRL) containing 10% fetal bovine serum (GIBCO-BRL) and adjusted to 3×10⁷ cells/mL. 200 μL of the KPMM2 cell suspension (6×10⁶ cells/mouse) was transplanted to the SCID mouse (male, 6 week-old) via caudal vein thereof, which had been hypodermically injected the asialo GM1 antibody (WAKO JUNYAKU, 1 vial, dissolved in 5 mL) on the day before the transplantation.

(4) Administration of Antibodies

The samples of the antibodies prepared in (2), the monomer (250 μL) and the dimer (400 μL), were administered to the model mice of human myeloma prepared in (3) via caudal vein thereof. The administration was started three days after the transplantation of KPMM2 cells and was carried out twice a day for three days. As a control, 200 μL of filtered PBS(−) was similarly administered twice a day for three days via caudal vein. Each group consisted of seven mice.

(5) Evaluation of Antitumor Effects of the Monomer and the Dimer of scFv/CHO Polypeptide for the Model Mouse of Human Myeloma

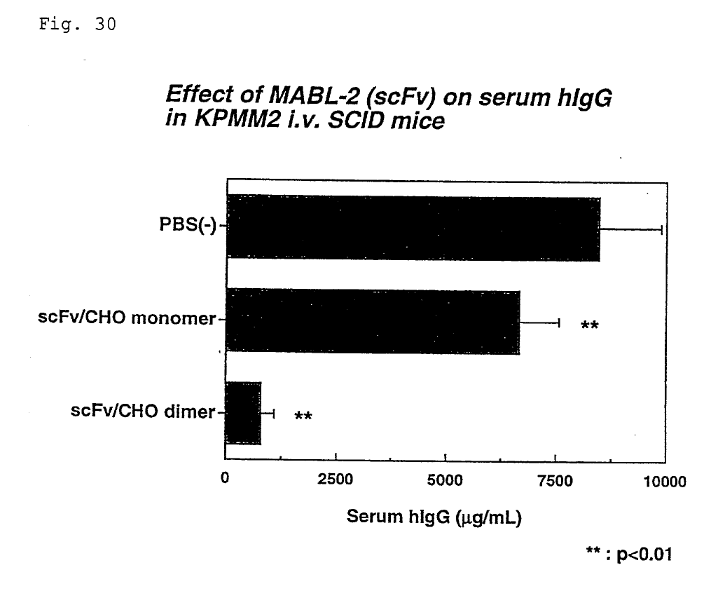

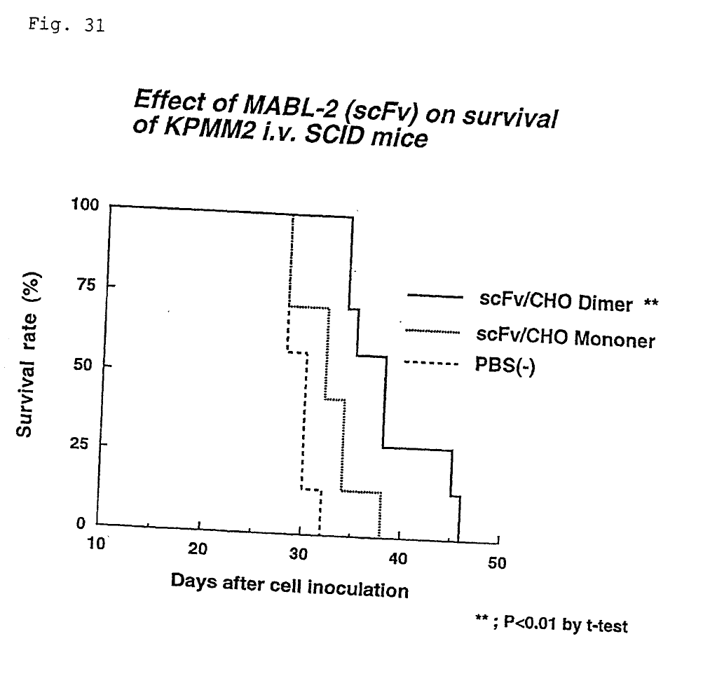

The antitumor effect of the monomer and the dimer of scFv/CHO polypeptide for the model mice of human myeloma was evaluated in terms of the change of human IgG content in the mouse serum and survival time of the mice. The change of human IgG content was determined as follows. The mouse serum was gathered 24 days after the transplantation of KPMM2 cells and an amount of human IgG in the serum was measured using the ELISA described in the above (1). As shown in FIG. 30, the amount of human IgG in the serum of the PBS(−)-administered group (control) increased to about 8500 μg/mL, whereas the amount of human IgG of the scFv/CHO dimer-administered group was remarkably low, that is, as low as one-tenth or less than that of the control group. Thus, the results suggest that the dimer of scFv/CHO could strongly inhibit the growth of the KPMM2 cells (FIG. 30). As shown in FIG. 31, a remarkable elongation of the survival time was observed in the scFv/CHO dimer-administered group in comparison with the PBS(−)-administered group.

From the above, it is confirmed that the dimer of scFv/CHO has an antitumor effect for the human myeloma model mouse. It is considered that the antitumor effect of the dimer of scFv/CHO, which is the reconstructed polypeptide according to the invention, results from the apoptosis inducing action of the reconstructed polypeptide.

5.15 Hemagglutination Test

Hemagglutination test and determination of hemagglutination were carried out in accordance with “Immuno-biochemical Investigation”, Zoku-Seikagaku Jikken Koza, Edited by the Biochemical Society of Japan, Tokyo Kagaku Dojin.

Blood was collected from healthy men using heparin-treated syringes and washed with PBS(−) three times, and then erythrocyte suspending fluid with a final concentration of 2% was prepared in PBS(−). Used as test samples were mouse IgG (ZYMED) as a control, the monomer and the dimer of the single-chain Fv polypeptide produced by the CHO cell, and the monomer and the dimer of the single-chain Fv polypeptide produced by E. coli. For the investigation of the hemagglutinatating effect, round bottom 96-well plates available from Falcon were used and 50 μL per well of the aforementioned antibody samples was added into each well. 50 μL of the 2% erythrocyte suspending fluid was added and mixed. After incubation for 2 hours at 37° C., the reaction mixtures were stored at 4° C. overnight and the hemagglutination thereof was determined. As a control, 50 μL per well of PBS(−) was added into each well and the hemagglutination test was carried out in the same manner. The mouse IgG and MABL-2 antibody were employed at 0.01, 0.1, 1.0, 10.0 or 100.0 μg/mL of the final concentration of the antibodies. The single-chain Fvs were employed at 0.004, 0.04, 0.4, 4.0, 40.0 or 80.0 μg/mL of the final concentration and 160.0 μg/mL of the dose was further designed only in the case of the dimer of the polypeptide produced by E. coli. Results are shown in the following table. In the case of MABL-2 antibody, the hemagglutination was observed at a concentration of more than 0.1 μg/mL, whereas no hemagglutination was observed for both the monomer and the dimer of the single-chain Fv.

TABLE 2
Hemagglutination Test

| | Control | 0.01 | 0.1 | 1 | 10 | 100 | μg/mL |
|---|---|---|---|---|---|---|---|
| mIgG | − | − | − | − | − | − | |
| MABL-2 | − | − | + | +++ | +++ | ++ | |
| (intact) | | | | | | | |

| | Control | 0.004 | 0.04 | 0.4 | 4 | 40 | 80 | μg/mL |
|---|---|---|---|---|---|---|---|---|
| scFv/CHO | − | − | − | − | − | − | − | |
| monomer | | | | | | | | |
| scFv/CHO | − | − | − | − | − | − | − | |
| dimer | | | | | | | | |

| | Control | 0.004 | 0.04 | 0.4 | 4 | 40 | 80 | 160 | μg/mL |
|---|---|---|---|---|---|---|---|---|---|
| scFv/E.coli | − | − | − | − | − | − | − | | |
| monomer | | | | | | | | | |
| scFv/E.coli | − | − | − | − | − | − | − | − | − |
| dimer | | | | | | | | | |

### EFFECT OF INVENTION

According to this invention, novel single-chain Fvs capable of inducing apoptosis of nucleated blood cells with Integrin Associated Protein (IAP) have the aforementioned amino acid sequences. The single-chain Fvs specifically recognize nucleated blood cells with human IAP and are capable of inducing apoptosis of the cells. Therefore, the single-chain Fvs of the invention are useful as a therapeutic agent for blood dyscrasia, for example, leukemia such as acute myeloid leukemia, chronic myelogenous leukemia, acute lymphoblastic leukemia, chronic lymphoblastic leukemia, adult T-cell leukemia, multiple myeloma, mixed leukemia and hairy cell leukemia, malignant lymphoma (Hodgkin's disease, non-Hodgkin's lymphoma), hypoplastic anemia, osteomyelodysplasia and polycythemia vera.

What is claimed is:

1. A polypeptide which is reconstructed with variable regions of the monoclonal antibodies capable of inducing apoptosis of nucleated blood cells having Integrin Associated Protein (IAP).

2. A DNA encoding a polypeptide of claim 1.

3. A single-chain Fv capable of inducing apoptosis of nucleated blood cells having Integrin Associated Protein (IAP).

4. An L chain V region comprising an amino acid sequence selected from
a) an amino acid sequence of SEQ ID No. 5: Met Lys Leu Pro Val Arg Leu Leu Val Leu Met Phe Trp Ile Pro Ala Ser Ser Ser Asp Val Val Met Thr Gln Thr Pro Leu Ser Leu Pro Val Ser Leu Gly Asp Gln Ala Ser Ile Ser Cys Arg Ser Ser Gln Ser Leu Leu His Ser Lys Gly Asn Thr Tyr Leu Gln Trp Tyr Leu Gln Lys Pro Gly Gln Ser Pro Lys Leu Leu Ile Tyr Lys Val Ser Asn Arg Phe Ser Gly Val Pro Asp Arg Phe Ser Gly Ser Gly Ser Gly Thr Asp Phe Thr Leu Lys Ile Ser Arg Val Glu Ala Glu Asp Leu Gly Val Tyr Phe Cys Ser Gln Ser Thr His Val Pro Tyr Thr Ser Gly Gly Gly Thr Lys Leu Glu Ile Lys;
b) an amino acid sequence of SEQ ID No. 7: Met Lys Leu Pro Val Arg Leu Leu Val Leu Met Phe Trp Ile Pro Gly Ser Ser Ser Asp Val Val Met Thr Gln Ser Pro Leu Ser Leu Pro Val Ser Leu Gly Asp Gln Ala Ser Ile Ser Cys Arg Ser Ser Gln Ser Leu Val His Ser Asn Gly Lys Thr Tyr Leu His Trp Tyr Leu Gln Lys Pro Gly Gln Ser Pro Lys Leu Leu Ile Tyr Lys Val Ser Asn Arg Phe Ser Gly Val Pro Asp Arg Phe Ser Gly Ser Gly Ser Val Thr Asp Phe Thr Leu Met Ile Ser Arg Val Glu Ala Glu Asp Leu Gly Val Tyr Phe Cys Ser Gln Ser Thr His Val Pro Tyr Thr Phe Gly Gly Gly Thr Lys Leu Glu Ile Lys; or
c) an amino acid sequence in which one or some amino acids are deleted, replaced or added into the amino acid sequence of a) or b).

5. An H chain V region comprising an amino acid sequence selected from
a) an amino acid sequence of SEQ ID No. 6: Met Glu Trp Ser Trp Ile Phe Leu Phe Leu Leu Ser Gly Thr Ala Gly Val His Ser Gln Val Gln Leu Gln Gln Ser Gly Pro Asp Leu Val Lys Pro Gly Ala Ser Val Lys Met Ser Cys Lys Ala Ser Gly Tyr Thr Phe Val Asn His Val Met His Trp Val Lys Gln Lys Pro Gly Gln Gly Leu Glu Trp Ile Gly Tyr Ile Tyr Pro Tyr Asn Asp Gly Thr Lys Tyr Asn Glu Lys Phe Lys Gly Lys Ala Thr Leu Thr Ser Glu Lys Ser Ser Ser Ala Ala Tyr Met Glu Leu Ser Ser Leu Ala Ser Glu Asp Ser Ala Val Tyr Tyr Cys Ala Arg Gly Gly Tyr Tyr Ser Tyr Asp Asp Trp Gly Gln Gly Thr Thr Leu Thr Val Ser Ser;
b) an amino acid sequence of SEQ ID No. 8: Met Glu Trp Ser Trp Ile Phe Leu Phe Leu Leu Ser Gly Thr Ala Gly Val His Ser Gln Val Gln Leu Gln Gln Ser Gly Pro Glu Leu Val Lys Pro Gly Ala Ser Val Lys Met Ser Cys Lys Ala Ser Gly Tyr Thr Phe Ala Asn His Val Ile His Trp Val Lys Gln Lys Pro Gly Gln Gly Leu Glu Trp Ile Gly Tyr Ile Tyr Pro Tyr Asn Asp Gly Thr Lys Tyr Asn Glu Lys Phe Lys Asp Lys Ala Thr Leu Thr Ser Asp Lys Ser Ser Thr Thr Ala Tyr Met Asp Leu Ser Ser Leu Ala Ser Glu Asp Ser Ala Val Tyr Tyr Cys Ala Arg Gly Gly Tyr Tyr Thr Tyr Asp Asp Trp Gly Gln Gly Thr Thr Leu Thr Val Ser Ser; or
c) an amino acid sequence in which one or some amino acids are deleted, replaced or added into the amino acid sequence of a) or b).

6. A DNA encoding the L chain V region of claim 4.

7. A DNA encoding the H chain V region of claim 5.

8. The DNA of claim 6 wherein the DNA encoding the L chain V region is selected from
a) a DNA of SEQ ID No. 5: atg aag ttg cct gtt agg ctg ttg gtg ctg atg ttc tgg att cct 45 gcg tcc agc agt gat gtt gtg atg acc caa act cca ctc tcc ctg 90 cct gtc agt ctt gga gat caa gcc tcc atc tct tgc aga tct agt 135 cag agc ctt cta cac agt aaa gga aac acc tat tta caa tgg tac 180 cta cag aag cca ggc cag tct cca aag ctc ctg atc tac aaa gtt 225 tcc aac cga ttt tct ggg gtc cca gac agg ttc agt ggc agt gga 270 tca ggg aca gat ttc aca ctc aag atc agc aga gtg gag gct gag 315 gat ctg gga gtt tat ttc tgc tct caa agt aca cat gtt ccg tac 360 acg tcc gga ggg ggg acc aag ctg gaa ata aaa c 394
b) a DNA of SEQ ID No. 7: atg aag ttg cct gtt agg ctg ttg gtg ctg atg ttc tgg att cct 45 ggt tcc agc agt gat gtt gtg atg acc caa agt cca ctc tcc ctg 90 cct gtc agt ctt gga gat caa gcc tcc atc tct tgc aga tca agt 135 cag agc ctt gtg cac agt aat gga aag acc tat tta cat tgg tac 180 ctg cag aag cca ggc cag tct cca aaa ctc ctg atc tac aaa gtt 225 tcc aac cga ttt tct ggg gtc cca gac agg ttc agt ggc agt gga 270 tca gtg aca gat ttc aca ctc atg atc agc aga gtg gag gct gag 315 gat ctg gga gtt tat ttc tgc tct caa agt aca cat gtt ccg tac 360 acg ttc gga ggg ggg acc aag ctg gaa ata aaa c 394 or
c) a DNA hybridizing to the DNA of a) or b) under the stringent condition.

9. The DNA of claim 7 wherein the DNA encoding the H chain V region is selected from
a) a DNA of SEQ ID No. 6: atg gaa tgg agc tgg ata ttt ctc ttc ctc ctg tca gga act gca 45 ggt gtc cac tcc cag gtc cag ctg cag cag tct gga cct gac ctg 90 gta aag cct ggg gct tca gtg aag atg tcc tgc aag gct tct gga 135 tac acc ttc gtt aac cat gtt atg cac tgg gtg aag cag aag cca 180 ggg cag ggc ctt gag tgg att gga tat att tat cct tac aat gat 225 ggt act aag tac aat gag aag ttc aag ggc aag gcc aca ctg act 270 tca gag aaa tcc tcc agc gca gcc tac atg gag ctc agc agc ctg 315 gcc tct gag gac tct gcg gtc tac tac tgt gca aga ggg ggt tac 360 tat agt tac gac gac tgg ggc caa ggc acc act ctc aca gtc tcc 405 tca g 409
b) a DNA of SEQ ID No. 8: atg gaa tgg agc tgg ata ttt ctc ttc ctc ctg tca gga act gca 45 ggt gtc cac tcc cag gtc cag ctg cag cag tct gga cct gaa ctg 90 gta aag cct ggg gct tca gtg aag atg tcc tgc aag gct tct gga 135 tac acc ttc gct aac cat gtt att cac tgg gtg aag cag aag cca 180 ggg cag ggc ctt gag tgg att gga tat att tat cct tac aat gat 225 ggt act aag tat aat gag aag ttc aag gac aag gcc act ctg act 270 tca gac aaa tcc tcc acc aca gcc tac atg gac ctc agc agc ctg 315 gcc tct gag gac tct gcg gtc tat tac tgt gca aga ggg ggt tac 360 tat act tac gac gac tgg ggc caa ggc acc act ctc aca gtc tcc 405 tca g 409 or
c) a DNA hybridizing to the DNA of a) or b) under the stringent condition.

10. The single-chain Fv of claim 3 which is a humanized single-chain Fv capable of inducing apoptosis of cells having Integrin Associated Protein (IAP).

11. The L chain V region of claim 4 which is a humanized L chain V region.

12. The H chain v region of claim 5 which is a humanized H chain V region.

13. A DNA encoding the humanized single-chain Fv of claim 10.

14. A humanized monoclonal antibody or a fragment thereof which can be prepared from a humanized single-chain Fv capable of inducing apoptosis of cells having Integrin Associated Protein (IAP).

15. A DNA encoding the humanized monoclonal antibody or the fragment thereof set forth in claim 14.

16. An animal cell which is capable of produing the single-chain Fv, the monoclonal antibody or the fragment thereof set forth in any one of claims 1, 3, 10 and 14.

17. A microorganism which is capable of produing the single-chain Fv, the monoclonal antibody or the fragment thereof set forth in any one of claims 1, 3, 10 and 14.

18. A therapeutic agent for blood dyscrasia comprising a substance capable of inducing apoptosis of cells having Integrin Associated Protein (IAP).

19. The therapeutic agent of claim 18 characterized in that the blood dyscrasia is leukemia.

20. The therapeutic agent of claim 18 characterized in that the substance is the single-chain Fv, the monoclonal antibody or the fragment thereof set forth in any one of claims 1, 3, 10 and 14.
